(19) United States
(12) Patent Application Publication
(10) Pub. No.: US 2009/0263329 A1
(43) Pub. Date: Oct. 22, 2009

(54) CELL LABELING WITH PERFLUOROCARBON NANOPARTICLES FOR MAGNETIC RESONANCE IMAGING AND SPECTROSCOPY

(75) Inventors: Samuel A. Wickline, St.Louis, MO (US); Gregory M. Lanza, St. Louis, MO (US)

Correspondence Address: THOMPSON COBURN LLP;ATTN: RICHARD E. HAFERKAMP, ONE U.S. BANK PLAZA, SAINT LOUIS, MO, 63101, US

(73) Assignee: Washington University, St., Louis, MO (US)

(21) Appl. No.: 12/280,396

(22) PCT Filed: Feb. 23, 2007
(86) PCT No.: PCT/US2007/004823

§ 371 Date: Feb. 12, 2009

Related U.S. Application Data
(60) Provisional application No. 60/776,743, filed on Feb. 24, 2006.

(51) Int. Cl. A61K 49/00; C12Q 1/02

(52) U.S. Cl. 424/9.37; 435/29

(57) ABSTRACT

Methods of obtaining cells internally labeled with perfluorocarbon nanoparticles suitable for magnetic resonance imaging and spectroscopy are disclosed. Also disclosed are methods for obtaining magnetic resonance imaging data from labeled under clinically relevant scan times and field strengths. Finally, the application further discloses methods of specifically detecting and distinguishing magnetic resonance imaging and spectroscopy data from two distinct sets of cells labeled with distinct types of perfluorocarbon nanoparticles.

### CROSS REFERENCE TO RELATED APPLICATIONS

This patent application claims priority to the Feb. 24, 2006 filing date of U.S. Provisional Patent Application No. 60/776,743, which is incorporated by reference herein in its entirety.

### STATEMENT REGARDING FEDERALLY SPONSORED RESEARCH OR DEVELOPMENT

National Institutes of Health (U54-CA-119342 and HL-073646 to S. A. Wickline, CO-37007 to G. M. Lanza).

### FIELD OF THE INVENTION

This invention relates generally to methods of obtaining labeled cells suitable for magnetic resonance imaging or magnetic resonance spectroscopy. The invention further relates to methods of magnetic resonance imaging or magnetic resonance spectroscopy that permit data acquisition from labeled cells under clinically relevant conditions (i.e., magnetic field strengths of 1.5 T with imaging times of less than about 12 minutes). Finally, this invention further provides for methods of obtaining two distinct magnetic resonance imaging or spectroscopy data sets derived from two distinct cells introduced into a system at the same time.

### RELATED ART

A variety of new disease treatments based on the use of living cells are currently under investigation. Disorders ranging from diabetes to various neural and cardiac diseases are currently targeted for treatment with cell-based therapies. Although cell based therapies promise to cure various diseases, initial experiments indicate that the fate and distribution of cells introduced into a host must be carefully monitored to insure that the introduced cells are functioning as intended. For example, it is critical to determine if the introduced cells are dividing, fusing with endogenous host cells, or are being destroyed through host immune responses. In therapies where the introduced cells must interact with endogenous host cells, it is crucial that the introduced cells be localized so as to permit appropriate interactions. This point is amply demonstrated in failed clinical trials where Parkinson's disease patients who had received transplants of neural cells exhibited involuntary movements as a result of establishing improper synaptic connections with resident cells (see article entitled “Proceed with Caution”, Nature Biotechnology 23(7):763, 2005). In therapies based on either complete or partial differentiation of the introduced cells, it is also critical to determine if the introduced cells are differentiating as intended rather than entering an unintended differentiation pathway that could lead to medical complications. This is especially important in the case of stem cell based therapies as stem cells have been reported to form teratomas (i.e., tumors comprised of unorganized masses of distinct cell types).

In considering methods of tracking cells introduced into a host for therapeutic purposes, a number of criteria are envisioned. First, it is important that the method of labeling and visualizing the introduced cells exert no deleterious effects on either the introduced cells themselves or upon the host into which they are placed. In this regard, it is important that the labeling and visualization method exert no effects that will significantly alter or compromise the intended biological function of the introduced cell. As supplies of the introduced cells may be limited, the labeling and visualization method should also conserve as many of the input cells as possible. Conservation of cells in the marking and visualization process is especially important in therapies based on reintroduction of cells derived from that same individual host (i.e., autologous cell transfer) where cell quantities are more limited. Second, the marking and visualization method must provide requisite levels of sensitivity and reproducibility. Ideally, the labeling and visualization would permit the localization and imaging of a single marked cell in a host organism. However, less sensitive methods providing for the localization of multiple cells in the host organism may also be useful in certain contexts. Third, the marking and visualization system must provide some level of persistence since the introduced cells are expected to exert their effects over an extended period of time. Finally, since the cell based therapies would ultimately be performed on patients in a clinical setting, the marking and imaging method would ideally be non-invasive and convenient.

Various means for tracking cells introduced into a host are currently available but do not meet the desired criteria of biological neutrality, conservation of input cells, sensitivity and clinically applicability. For example, a large number of techniques based on “targeted” cell labeling with various receptor binding ligands or antibodies are known in the art (for example see U.S. Pat. No. 6,676,963). A primary concern with using ligands that bind to cell surface receptors or antibodies that bind to cell surface proteins is that such binding will compromise the biological functions of the targeted cells. This concern is not unfounded as the cell surface receptors mediate a variety of biological processes by interacting with naturally occurring ligands. Similarly, the functions of other cell surface proteins may also be compromised by antibody binding. Yet another concern with targeted labeling techniques is that it may be necessary to use antibodies directed to relatively abundant cell surface proteins in order to obtain sufficiently sensitive levels of cell detection.

One “non-targeted” cell tracking approach entails use of metal-ion based ¹H contrast agents such as super paramagnetic iron oxide (SPIO) nanoparticles or gadolinium-based T₁ agents coupled with proton (¹H) Magnetic Resonance Imaging (MRI). (Yeh et al., Magn. Reson. Med 30: 617-625, 1993; Bulte et al., Method. Enzymol. 386: 275-299, 2004). However, such methods suffer from the inherent ambiguity associated with distinguishing the ¹H signal associated with the labeled cells from the background ¹H signal associated with mobile water. These particles are typically introduced into cells isolated in vitro after treatment with transfection agents such as cationic lipids, or with mechanical approaches such as electroporation. Such methods used in conjunction with iron oxide particles can lead to significant losses in cell viability. To circumvent cell viability issues associated with use of transfection reagents, de Vries et al. (Nature Biotechnol. 23 (11): 1407-1413, 2005) co-cultured highly phagocytic immature dendritic cells with SPIO nanoparticles and were able to detect the labeled dendritic cells in both patients and in tissues harvested from the patients. However, it is not clear that cells that are less phagocytic than immature dendritic cells would internalize sufficient amounts of nanoparticles to permit imaging. For example, transfection reagents have typically been used to label less phagocytic stem cells (Hoehn et al., Proc. Natl. Acad. Sci. USA 99: 16267-16272, 2002), and it has been reported that stem cells take up negligible amounts of nanoparticles when transfection reagents are not employed (Frank et al. Radiology 228:480-487, 2003).

Ahrens et al. labeled dendritic cells with perfluoro-15-crown-5 ether nanoparticles by use of cationic lipid transfection reagents and imaged the cells by use of ¹⁹F MRI (Magnetic Resonance Imaging; Ahrens et al., Nature Biotechnol. 23(8):983-987, 2005). This method obviates the signal-to-background problems associated with ¹H MRI as living tissues have very low ¹⁹F background levels. However, this particular study was only able to demonstrate detection of the ¹⁹F labeled cells through use of a powerful 11.7 T field strength MRI instrument and required about 3 hours of imaging. These conditions are clearly impractical in a clinical setting where patients would be imaged. Moreover, the use of a cationic transfection reagents is known to result in the loss of cell viability. In Ahrens et al. (Ibid), only the cells that survived treatment with the cationic transfection reagent were tested for effects on cell viability. The percentage of the total input cells surviving the cationic transfection reagent treatment was not provided by Ahrens et al. Finally, this method only allows for detection of one labeled cell type at any given point in time.

### SUMMARY OF THE INVENTION

It is in view of the above problems that the present invention was developed. The invention is first drawn to a method of obtaining an endothelial precursor cell suitable for magnetic resonance imaging or spectroscopy comprising the steps of providing an endothelial precursor cell; incubating said endothelial precursor cell in a cell culture media containing a plurality of perfluorocarbon nanoparticles for a period of time and at a perfluorocarbon nanoparticle concentration sufficient to result in internalization of a detectable level of perfluorocarbon nanoparticles; and separating said endothelial precursor cell from said culture media containing perfluorocarbon nanoparticles. The perfluorocarbon nanoparticles comprise a perfluorooctylbromide core component or a perfluoro-15-crown-5-ether core component. When the perfluorocarbon nanoparticles comprise a perfluorooctylbromide core component, a detectable level of internalized perfluorocarbon nanoparticles is an intracellular perfluorocarbon nanoparticle concentration of at least 2.8 pmol per cell. When the perfluorocarbon nanoparticles comprise a perfluoro-15-crown-5-ether core component, a detectable level of internalized perfluorocarbon nanoparticles is an intracellular perfluorocarbon nanoparticle concentration of at least 0.5 pmol per cell. The endothelial precursor cell may be provided by isolating mononuclear cells from human umbilical cord blood and growing the cells in a modified endothelial cell culture media. This modified endothelial cell culture media may comprise the growth factors hEGF, VEGF, hFGF-B, and R3-IGF-1. The endothelial precursor cell may be any one of a CD34+ cell, CD133+ cell, CD31+ cell, a Tie-2+ cell, a CD31+/CD34+ cell, CD34+/CD133+/CD31+ cell, a CD34+/Tie-2+ cell, a CD34⁺CD133⁺Tie-2⁺CD45⁺ cell, and a CD34+/CD133+ cell. Alternatively, the endothelial precursor cell may be characterized by an ability to internalize acetylated-Low Density Lipoprotein (LDL) and/or by the presence of fucose at its surface. The endothelial precursor cell suitable for magnetic resonance imaging that is obtained by this method can typically internalize acetylated-Low Density Lipoprotein (LDL) and has fucose present at its surface. By using this method, one skilled in the art can obtain an endothelial precursor cell suitable for magnetic resonance imaging without using methods such as electroporation or transfection to introduce the perfluorocarbon nanoparticles into the cells. The advantages of using this technique are that introduction of the perfluorocarbon nanoparticles has minimal effects on cell viability and minimizes loss of input cells.

This invention is further drawn to a method for obtaining a magnetic resonance image of a plurality of cells introduced into a subject at a magnetic field strength of 1.5 T comprising the steps of: a) obtaining a plurality of cells with an intracellular perfluoro-15-crown-5-ether nanoparticle concentration of at least 0.5 pmol per cell; b) introducing said plurality of cells from step (a) into a subject; c) exposing said subject from step (b) to a magnetic field strength of 1.5 T; and d) obtaining magnetic resonance image data via a magnetic resonance imaging method, thereby obtaining a magnetic resonance, image of a plurality of cells introduced into a subject. In practicing this method, perfluoro-15-crown-5-ether nanoparticles may be introduced into the cells by a method such as electroporation, transfection, ultrasound, or sonication. Alternatively, the perfluoro-15-crown-5-ether nanoparticles may be introduced into the cells such as endothelial precursor cells by providing an endothelial precursor cell, incubating the endothelial precursor cell in a cell culture media containing perfluoro-15-crown-5-ether nanoparticles for a period of time and at a perfluorocarbon nanoparticle concentration sufficient to result in an intracellular perfluorocarbon nanoparticle concentration of at least 0.5 pmol and separating said endothelial precursor cell from said culture media containing perfluorocarbon nanoparticles. This method may be practiced on subjects that are mammals such as a mouse, a rat, a rabbit, a cat, a dog, a pig, a cow, a horse, a monkey, or a human. The particular magnetic resonance imaging method may comprise any of a steady state free precession pulse sequence (SSFP), a balanced-fast field echo imaging sequence or a SSFP-fast field echo imaging sequence. One set of suitable magnetic resonance imaging conditions for practicing this method would be a balanced fast field echo imaging sequence comprising an echo time (TE) of 5 ms, a time to repetition (TR) of 10 ms, 512 signal averages, a 2.5×2.5 mm reconstructed in-plane resolution, a 60 degree flip angle, a 35 mm slice thickness, and a total scan time of between 2 to 10 minutes. Advantages of this imaging method are that the subject is exposed to a magnetic field of reduced strength and that the image is acquired in a shorter period of time.

The invention is also drawn to methods of obtaining two distinct magnetic resonance spectroscopy or magnetic resonance imaging data sets derived from two distinct cells introduced into a system, comprising the steps of:

(a) obtaining a first cell containing a first intracellular perfluorocarbon nanoparticle, wherein said first intracellular perfluorocarbon nanoparticle comprises a perfluoro-15-crown-5-ether core component and wherein said first intracellular perfluorocarbon nanoparticle is at a detectable level in said first cell;

(b) obtaining a second cell containing a second intracellular perfluorocarbon nanoparticle, wherein said second intracellular perfluorocarbon nanoparticle comprises a perfluorooctylbromide core component and wherein said second intracellular perfluorocarbon nanoparticle is at a detectable level in said second cell;

(c) introducing said first cell from step (a) and said second cell from step (b) into a system;

(d) exposing said system from step (c) to a first magnetic field and obtaining magnetic resonance spectroscopy or magnetic resonance imaging data for said first cell with a magnetic resonance spectroscopy method or magnetic resonance imaging method that specifically detects a first ¹⁹F MRI signal from said perfluoro-15-crown-5-ether core component to obtain a first spectroscopy data set or a first imaging data set from said first cell;

e) exposing said system from step (c) to a second magnetic field and obtaining magnetic resonance spectroscopy data or magnetic resonance imaging data for said second cell with a magnetic resonance spectroscopy method that specifically detects a second ¹⁹F MRI signal from said perfluorooctylbromide core component to obtain a second spectroscopy data set or a second imaging data set from said second cell, thereby obtaining two distinct magnetic resonance spectroscopy data sets or two distinct magnetic resonance imaging sets derived from two distinct cells introduced into a system. Cells containing detectable levels of either the first or second PFC nanoparticles can be obtained by separately introducing said perfluorocarbon nanoparticles by electroporation, transfection, ultrasound, and sonication. When the perfluorocarbon nanoparticle comprises a perfluoro-15-crown-5-ether core component, a detectable level in a cell is an intracellular concentration of this perfluorocarbon nanoparticle that is at least 0.5 pmol per cell. When the perfluorocarbon nanoparticle comprises a perfluorooctylbromide core component, a detectable level in a cell is an intracellular concentration of this perfluorocarbon nanoparticle is at least 2.8 pmol per cell. Alternatively, endothelial precursor cell with detectable levels of PFC nanoparticles can be obtained by incubating said endothelial precursor cell in a cell culture media containing a plurality of a first perfluorocarbon nanoparticle comprising either a perfluoro-15-crown-5-ether core or a perfluorooctylbromide core component for a period of time and at a perfluorocarbon nanoparticle concentration sufficient to result in internalization of a detectable level of the perfluorocarbon nanoparticle concentration and separating said endothelial precursor cell from said culture media containing perfluorocarbon nanoparticles. In this method, imaging or spectroscopy data from two distinct cells may be acquired from cells introduced into a system that is an in vitro system. This in vitro system may be a system of regenerating a tissue or an organ outside of a host organism. The in vitro system may also be a test tube, a petri dish, a microtiter plate well, a roller bottle, and a cell culture reactor. Alternatively, the system may be a living organism. The living organism may be a mammal. This mammal may be a mouse, a rat, a rabbit, a cat, a dog, a pig, a cow, a horse, a monkey, or a human. The magnetic fields applied may have a field strength of 11.7 T. Alternatively, the magnetic fields applied may have a field strength of 1.5 T.

To practice methods of obtaining two distinct magnetic resonance imaging data sets derived from two distinct cells introduced into a system, methods of specifically detecting and distinguishing ¹⁹F MRI signals derived from either a perfluoro-15-crown-5-ether core component or a perfluorooctylbromide core components are taught. These magnetic resonance imaging methods comprise use of an excitation signal centered at a frequency that is substantially the same as the resonance frequency of one PFC nanoparticle core component (i.e., such as when the PFC nanoparticle is perfluoro-15-crown-5-ether), where the excitation signal bandwidth does not overlap any of several resonance frequencies of the other PFC nanoparticle core component (i.e., such as for a perfluorooctylbromide core component). In this case (i.e., when specifically detecting perfluoro-15-crown-5-ether core components), this excitation signal is typically a narrow bandwidth excitation signal. This narrow bandwidth excitation signal may have a bandwidth of 1002 Hz. When specifically detecting a perfluorooctylbromide core component, this method comprises use of an excitation signal centered at a frequency that is substantially the same as the resonance frequency of at least one selected spectral peak generated by the perfluorooctylbromide core component, wherein said excitation signal bandwidth does not overlap the resonance frequency of said perfluoro-15-crown-5-ether core components. Again, this excitation signal is typically a narrow bandwidth excitation signal. One such method for specifically detecting a perfluorooctylbromide core component is to use a narrow bandwidth excitation signal that has a bandwidth of 900 Hz for a single resonance peak for said perfluorooctylbromide core component that is 600 Hz removed from a perfluoro-15-crown-5-ether resonance peak. Another such method for specifically detecting a perfluorooctylbromide core component is to use a narrow bandwidth excitation signal that has a bandwidth of 2018 Hz for a plurality of resonance peaks for the perfluorooctylbromide core component that are 2000 Hz removed from a perfluoro-15-crown-5-ether resonance peak.

To practice methods of obtaining two distinct magnetic resonance spectroscopy data sets derived from two distinct cells introduced into a system, methods of specifically detecting and distinguishing ¹⁹F MRI signals derived from either a perfluoro-15-crown-5-ether core component or a perfluorooctylbromide core components are also taught. These methods for obtaining magnetic resonance spectroscopy data for a single PFC nanoparticle core component (i.e., either a perfluoro-15-crown-5-ether core component or a perfluorooctylbromide core component) comprise acquisition of volume selective spectra by image-selective in vivo spectroscopy.

Methods of obtaining a monocyte cell suitable for magnetic resonance imaging are also provided by this invention. Such methods comprise the steps of providing an monocyte cell, incubating the monocyte cell in a cell culture media containing a plurality of perfluorocarbon nanoparticles for a period of time and at a perfluorocarbon nanoparticle concentration sufficient to result in internalization of a detectable level of perfluorocarbon nanoparticles and separating the monocyte cell from the culture media containing perfluorocarbon nanoparticles. In this method, the monocyte cell can be derived from blood or from bone marrow. The period of incubation of the monocytes with the PFC nanoparticles is at least about 3 hours. However, it is also contemplated that sufficient labeling periods of up to about 3 hours can be identified.

Methods of rapidly obtaining a cell suitable for magnetic resonance imaging with non-targeted perfluorocarbon nanoparticles and ultrasound energy are also provided. Such methods for obtaining a cell suitable for magnetic resonance imaging comprise the steps of providing at least cell, treating the cell or cells in a cell culture media containing a plurality of non-targeted perfluorocarbon nanoparticles with ultrasound energy for a period of time and at a perfluorocarbon nanoparticle concentration sufficient to result in internalization of a detectable level of perfluorocarbon nanoparticles; and separating the cell or cells from the culture media containing perfluorocarbon nanoparticles. In this method, the cell (or cells) can be an endothelial precursor cell or cells or a monocyte cell or cells. The non-targeted perfluorocarbon nanoparticles used in this method do not comprise nor require targeting molecules such as antibodies or receptor binding proteins in order to be internalized. Ultrasound energy can be applied at a frequency of at least about 1 to about 3 MHz and at power levels of at least about 0.5 to about 1.9 MI (MI: the machine power output setting) in a conventional imaging device. Conventional imaging devices that can be used in this method include devices such as the Siemens Sequoia™, Philips iE33™, GE LogiQ™, or any other mechanically scanned or array based systems platform or handheld scanner. Any other custom designed ultrasound imaging or therapeutic systems using transducers that can deliver focused energy can also be used in this method. Ultrasound exposure periods of between about 1 and about 15 minutes per ultrasound delivery field are sufficient to label cells with the nanoparticles. An ultrasound delivery field is the area within which ultrasound energy sufficient to result in nanoparticle internalization is provided. Cells distributed across a surface that is larger than the ultrasound delivery field are treated by delivery of ultrasound energy to a plurality of fields within said surface. This is accomplished by moving the ultrasound probe across the surface for a period of time that will permit delivery of ultrasound energy to the plurality of fields. Movement of the probe across the surface can be either continuous or accomplished in discrete steps. Coupling the ultrasound transducer to a movement device permits delivery of ultrasound to cells distributed across a surface such as a cell culture plate, a standard lab Petri dish, a 4-, 6-, 12-, 96-, 384-, or 1536-well microtiter plate, an Opticell membrane system, or other cell support. When the cells are distributed in distinct wells of a microtiter plate, ultrasound energy can be delivered to individual wells of the microtiter plate. The concentration of perfluorocarbon nanoparticles used in the treatment step is at least about 30 pM.

Further features and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1: Internalization of PFC nanoparticles. Confocal micrographs with simultaneous DIC imaging show, when compared to controls, cells contain significant amounts of either NBD-labeled perfluorooctyl bromide (PFOB) nanoparticles or rhodamine-labeled crown ether (CE) nanoparticles. Nanoparticles are localized to the cell cytosol and not the plasma membrane or nucleus. The scale bar represents 5 μm.

FIG. 2: Cellular Immunophenotyping Determinations. Flow cytometry dot plots of signal intensity of an array of monoclonal antibodies used to determine markers expressed by unloaded and NP-loaded cells. Panels (a-c) show results before incubation with nanoparticles where the majority of the population appears to be CD34⁺CD133⁺Tie-2⁺CD45⁺ with smaller portions of CD34⁺CD133⁻ cells (panel a), CD34⁺Tie-2⁻ cells (panel b), and CD133⁺CD45⁻ (panel c) within the population. Panels (d-f) show results for the population of cells containing nanoparticles (d), population of cells containing nanoparticles that stain positive for CD31 (e) and population of cells containing nanoparticles that stain positive for CD31 and CD34 (f).

FIG. 3: Cellular viability and function unaffected by nanoparticles. a) Exposure of cells to a 30 picomolar concentration of PFOB or CE nanoparticles (NPs) resulted in no significant change in cell viability from controls (˜90%, see dashed line). For positive controls, cells exposed to high temperature showed a significant loss in cell viability (error bars represent SEM). b) Flow cytometry dot plot of NP-loaded cells shows cells internalize acetylated LDL and stain positive for UEA-1. c) Confocal micrographs of DiI-acLDL inside endothelial and monocytic lineage cells and FITC-UEA-1 on membranes of endothelial lineage cells.

FIG. 4: 12 T MR spectroscopy, imaging, and quantification in vitro. Analysis of CE loaded (top) and PFOB loaded cells (bottom) reveals the ability to obtain spectrum, images, and PFC levels from nanoparticle-loaded cells. a) MR spectrum (˜2 min acquisition) showing one CE peak (arrow) and five PFOB peaks (all others) originating from loaded cells or nanoparticle standard. b) High resolution MR images (˜7 min scan time) of cross sections through cell pellets (˜4×10⁵ cells). c) MR quantification of PFC concentrations achieved per cell due to nanoparticle loading (error bars represent SEM).

FIG. 5: 1.5 T in vitro imaging of labeled cells. Proton (¹H) and fluorine (¹⁹F) projection images of CE-loaded cell pellets (˜4×10⁵ cells) from top to bottom. “Treated cells” were incubated with nanoparticles for 12 hours, while “control cells” were only incubated for ˜10 minutes revealing the specificity of image detection for loaded cells.

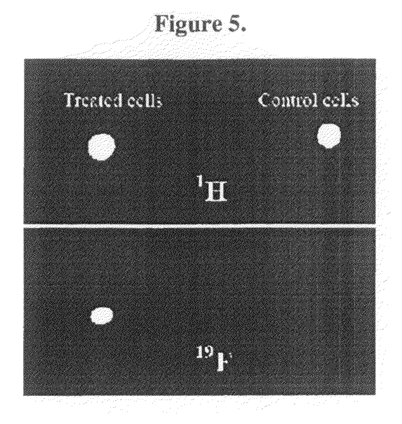

FIG. 6: Localization of labeled cells after in situ injection. Overlay of high resolution ¹⁹F images (˜7 min) onto conventional ¹H images (˜3 min) permits rapid, exact location of injected cells. At 12 T signal due to ˜1×10⁶ CE-loaded cells (a) when overlaid onto ¹H image of the site reveals the cell location to be in the mouse muscle near the femur and adjacent to an air void introduced during injection (b). Similarly at 1.5 T, ¹⁹F image of ˜4×10⁶ CE-loaded cells (c) locates to the mouse thigh in a ¹H image of the mouse cross-section. The absence of background signal in 19F images (a,c) enables unambiguous localization of perfluorocarbon containing cells at both 12 T and 1.5 T.

FIG. 7: Perfluorocarbon Nanoparticles. In the present experiments described in Example 7, two different liquid perfluorocarbon nanoparticles were used. One incorporated perfluoro-15-crown-5-ether (CE) and the other perfluorooctylbromide (PFOB), thus each having a unique fluorine signature. For targeting to fibrin through an anti-fibrin antibody, each of the nanoparticles had biotin (⋄) anchored onto the lipid monolayer surrounding the core. Additionally, the CE nanoparticles were made paramagnetic by attaching approximately 90,000 Gd-DTPA-bis-oleate molecules () onto the outer surface.

FIG. 8: Fluorine Transmit/Receive Coil. A single turn solenoid coil, shown here from two perspectives, was built in-house and tuned to the resonance frequency of ¹⁹F. This coil was large enough to house the 12-well plate completely and provides uniform fields for both transmit and receive modes.

FIG. 9: T1-Weighted Imaging of Fibrin Clots. (A) A single slice from a high-resolution T1-weighted acquisition acquired perpendicular to the plane of the clots. Paramagnetic nanoparticles, bound to the clots in cross section, appear as a bright line of signal enhancement with intensity decreasing linearly as the concentration of paramagnetic nanoparticle decreases (left to right). Performing a maximum intensity projection through the 3D data depicts the clots en face (B).

FIG. 10: ¹⁹F Spectra Acquired from Fibrin Clots. These spectra show the signal received from all 12 clots (i.e., no volume selection). Both types of nanoparticles, crown ether and perfluorooctylbromide (PFOB), can be detected and independently resolved. Performing multiple signal averages gives a high signal-to-noise spectrum (A), whereas a one minute acquisition (B) still provides unique recognition of the perfluorocarbons.

FIG. 11: Volume Selective Spectra from Fibrin Clots. Using volume selective spectroscopy to isolate the groups of clots with the various concentrations of perfluorocarbons, one can detect the changing ¹⁹F signature as the concentration of crown ether (CE) decreases inversely with perfluorooctylbromide (PFOB). Beginning with the first (bottom) spectra to the last (top), the relative concentrations of CE:PFOB are 1:0, 2:1, 1:2, and 0:1, respectively.

FIG. 12: ¹⁹F MR Imaging of Liquid Perfluorocarbon (PFC) Nanoparticles Bound to Fibrin Clots. These three fluorine images, which have no proton “background,” are oriented perpendicular to the fibrin clots as in FIG. 2 and were each acquired in under three minutes using steady state gradient echo techniques. They represent three “weightings” of the same imaging slice and illustrate the fibrin-bound PFC nanoparticles of various mixtures (i.e., crown ether (CE) or perfluorooctylbromide (PFOB)) applied to the clots. By using a broad bandwidth excitation (A), all fibrin clots enhance brightly regardless of the type of PFC nanoparticle bound to it. Employing a narrow bandwidth excitation corresponding to the resonance frequency of CE (B) allows independent visualization of the clots with CE nanoparticles bound. Likewise, selective excitation for PFOB provides independent imaging of PFOB nanoparticles (C). Note also the linear relationship between signal intensity and the relative concentration of each perfluorocarbon (as labeled above each clot in panel A).

FIG. 13: ¹⁹F Image-based Quantification of PFC Nanoparticle Concentration. Region of interest analysis on the ¹⁹F images allows relative quantification of perfluorocarbon concentration. Based on the perfluorocarbon-selective image (crown ether (CE) or perfluorooctylbromide (PFOB)), the signal intensity changes linearly with relative concentration of fibrin-bound nanoparticle.

FIG. 14: In vitro Imaging of Human Endarterectomy Specimen. (A) The proton (¹H) images on the left illustrate, in liquid-filled test tubes, two carotid artery specimen from symptomatic patients. Targeting crown ether (CE) nanoparticles to fibrin demonstrates the detection of exposed fibrin on ruptured plaque in the carotid artery. The ¹⁹F images show the “hot spots” of bound nanoparticles (in the same orientation as the proton image, dotted lines represent edges of the test tubes). (B) Combining the ¹⁹F and ¹H images allows better localization of the bound nanoparticles.

FIG. 15: Analysis of PFC Nanoparticle Labelled Peripheral Blood Monocytes by FACS. FACS analysis shows that 42% of F4/80+ peripheral blood mononuclear cells are labeled at 3 hrs. After the 3 hour labeling, 7.25% of total cells are positive for both the PFC nanoparticles and the F4/80 monocyte specific marker, while 25% of the total treated cells are positive for the PFC nanoparticles.

FIG. 16: Analysis of PFC Nanoparticle Labelled Bone Marrow Monocytes by FACS. FACS analysis shows that 17% of CD11b+, and 15% of Gr-1+ cells were PFC nanoparticle positive (+) at 3 hrs. About 9% and 8% of total cells are positive for both the PFC nanoparticles and each marker, respectively. Fifteen percent of the total treated bone marrow cells are positive for the PFC nanoparticles.

FIG. 17: Comparison of Cell Labeling Efficiency in the Presence and Absence of Ultrasound Treatment. The percentage of PFC nanoparticle labeled cells obtained after treatment for either: a) 12 hours without ultrasound (12 hrs, −US), b) 1 hour without ultrasound (1 hr, −US), and c) 1 hour with ultrasound (1 hr, +US).

### DETAILED DESCRIPTION OF THE PREFERRED EMBODIMENTS

### Definitions

The term “endothelial precursor cell” as used in this application refers to any one of or any combination of:

a) a cell characterized by the presence of any one of the following cell-surface markers or combinations of cell surface markers at the cell's surface: CD34+, CD133+, CD31+, Tie-2+ cell, CD31+/CD34+, CD34+/CD133+/CD31+, a CD34+/Tie-2+, CD34⁺CD133⁺Tie-2⁺CD45⁺ cell, and a CD34+/CD133+; and/or

b) a cell characterized by the presence of fucose at its surface; and/or

c) a cell characterized by its ability to internalize acetylated LDL (low density lipoprotein).

The term “perfluorocarbon nanoparticle” as used in this application refers to a nanoparticle comprising a perfluorocarbon core component such as perfluorooctylbromide or perfluoro-15-crown-5-ether that is coated with a mixture comprising various combinations or proportions of lipids, sterols, glycerin and/or surfactants.

The term “perfluorooctylbromide core component” as used in this application refers that portion of a perfluorocarbon nanoparticle that is primarily composed of perfluorooctylbromide. The acronym “PFOB” is also used herein to describe this core component.

The term “perfluoro-15-crown-5-ether core component” as used in this application refers that portion of a perfluorocarbon nanoparticle that is primarily composed of perfluoro-15-crown-5-ether. The acronym “CE” is also used herein to describe this core component.

### Methods of Obtaining an Endothelial Precursor Cell Suitable for Magnetic Resonance Imaging

Certain methods of this invention are directed to obtaining endothelial precursor cell suitable for magnetic resonance imaging by providing conditions that permit the cells to internalize the perfluorocarbon nanoparticles provided in the cell culture media. By employing the steps described in this method, it is possible to obtain populations of endothelial precursor cells suitable for magnetic resonance imaging without using transfection reagents or electroporation.

To practice this method, mononuclear cells are first isolated from umbilical cord blood and grown in modified endothelial growth media. A variety of modified endothelial growth media have been described and can be used for in this method. For example, Terramini et al. describe various growth media formulations suitable for growth of Human Umbilical Vein Endothelial cells (Terramini et al. In vitro Cell. Dev. Biol. Anim. 36(2):125-132, 2000), any one of which may be suitable for the use in the method described herein. For example, an optimized modified endothelial growth media described by Terramini et al. that is potentially useful in the practice of this invention comprises a MCDB 131 basal media (Gibco BRL, Gaithersberg, Md., USA) supplemented with Fetal Bovine Serum (FBS) at a concentration of 20%, Endothelial Cell Growth Supplement at a concentration of 50 ug/ml, Heparin at a concentration of 50 ug/ml, and 2 mM glutamine that is optionally substituted with the antibiotics amphotericin (2.5 ug/ml), penicillin (50 U/mL), and streptomycin (50 ug/mL). Another particularly useful modified endothelial growth media disclosed herein as useful in the practice of this invention comprises modified endothelial growth media (Clonetics EGM™-2-Endothelial Growth Medium-2; Cambrex, East Rutherford, N.J.) adjusted to a final concentration 20% FBS (Fetal Bovine Serum). The EGM™-2 modified endothelial growth media is comprised of human epidermal growth factor (hEGF), vascular endothelial growth factor (VEGF), human fibroblast growth factor (hFGF-B), and R3-insulin-like growth factor I (R3-IGF-I) as well as other components such as heparin, ascorbic acid and hydrocortisone.

The endothelial precursor cells are typically grown on cell culture plates coated with an extracellular matrix protein (ECM). Growth of the cells as adherent monolayers on the plates facilitates steps of the method such as providing an endothelial precursor cell, incubating endothelial precursor cell in a cell culture media containing perfluorocarbon nanoparticles, and separating the endothelial precursor cells from culture media containing perfluorocarbon nanoparticles. As discussed in Terramini et al. (ibid), it is anticipated that a variety of ECM proteins such as laminin, fibronectin, gelatin or collagen types I or type IV can be used to coat the plates used to grow both originally isolated HUVEC cells and the endothelial precursor cells as described in this method. In the non-limiting example provided herein, the plates used to grow the HUVEC cells and the endothelial precursor cells are coated with fibronectin. Cells can also be detached from the ECM coated plates by a variety of methods comprising use of enzymes such as trypsin. Alternatively, a non-enzymatic cell dissociation solution based on use of chelating agents that remove divalent cations can be used. In the example provided here a cell dissociation solution comprising EDTA, glycerol, and sodium citrate in a suitable buffer such as Hank's balanced salts or phosphate buffered saline (Sigma, St. Louis, Mo., USA) is used.

In practicing this invention, it is necessary to incubate the endothelial precursor cell in a cell culture media containing a plurality of perfluorocarbon nanoparticles for a period of time and at a perfluorocarbon nanoparticle concentration sufficient to result in internalization of a detectable level of perfluorocarbon nanoparticles. Having established a detectable level of a given type of PFC nanoparticle, the time periods and concentrations of PFC nanoparticles that are sufficient to result in a detectable level of the internalized PFC nanoparticles can easily be determined by empirical experimentation. For example, aliquots of the provided endothelial cells may be distributed to multiple wells of a microtiter plate and incubated at different PFC nanoparticle concentrations and/or for different periods of times, separated from un-internalized PFC nanoparticles and harvested at various time points, and then subjected to the magnetic resonance imaging methods described herein to determine if detectable levels of the PFC nanoparticle have been internalized.

In the particular examples disclosed herein, a concentration of at least about 30 pM (pico Molar) of the PFC nanoparticles in the cell culture media is sufficient to result in internalization of a detectable level of perfluorocarbon nanoparticles by the endothelial precursor cells. However, it is understood from the foregoing discussion that other concentrations of PFC nanoparticles in the cell culture media that result in internalization of PFC nanoparticles sufficient to permit magnetic resonance imaging or spectroscopy can be easily determined. Thus, PFC nanoparticle concentrations of at least about 25, 20, 10 or 5 pM may be tested and determined to result in internalization of a detectable level of perfluorocarbon nanoparticles. Moreover, concentrations of PFC nanoparticles of at least about 25, 20, 10 or 5 pM could also be used without departing from the essential thrust of this invention.

In the particular examples disclosed herein, a incubation time of at least about 12 hours of the endothelial precursor cells in the cell culture media is sufficient to result in internalization of a detectable level of perfluorocarbon nanoparticles by those cells. However, it is also shown that an exceedingly short time period of incubation (i.e., 10 minutes) is not sufficient to result in internalization of a detectable level of perfluorocarbon nanoparticles by those cells under the incubation conditions taught herein. Nonetheless, it is apparent from the foregoing discussion that time periods of less than 12 hours but more than 10 minutes of incubation in the cell culture media that result in internalization of PFC nanoparticles sufficient to permit magnetic resonance imaging or spectroscopy can be easily determined by practicing the methods taught in this instant invention. Thus, time periods of incubation of at least about 10, 8, 6, 4, 2 or 1 hours may be tested and determined to result in internalization of a detectable level of perfluorocarbon nanoparticles under the incubation conditions taught herein. Consequently, incubation time periods of at least about 10, 8, 6, 4, 2 or 1 hours could be used under the incubation conditions described herein without departing from the essential thrust of this invention. It is further understood that incubation conditions described in this paragraph are the conditions described herein that do not entail use of electroporation, transfection, ultrasound, and sonication methods to attain internalization of a detectable level of perfluorocarbon nanoparticles. One skilled in the art would understand that shorter incubation periods could be used in conjunction with distinct methods of introducing perfluorocarbon nanoparticles into cells (i.e., use of electroporation, transfection, ultrasound, and sonication methods to introduce the perfluorocarbon nanoparticles) to attain internalization of a detectable level of perfluorocarbon nanoparticles.

Finally, it is further noted that both the PFC nanoparticle concentration and time periods of incubation may be systematically varied in relation to one another to identify various combinations of PFC nanoparticle concentrations and incubation times that result in internalization of PFC nanoparticles sufficient to permit magnetic resonance imaging or spectroscopy. For example, cells could be incubated in the presence of PFC nanoparticle concentrations of between at least 30 pM to at least about 5 pM for times ranging between at least about 1 to 12 hours to identify various combinations of PFC nanoparticle concentrations and incubation times sufficient to result in internalization of PFC nanoparticles sufficient to permit magnetic resonance imaging or spectroscopy.

It is shown herein that a detectable level of internalized PFC nanoparticles is at least 2.8 pmol per cell of a PFC nanoparticle comprising a perfluorooctylbromide core component. It is also shown herein that a detectable level of internalized PFC nanoparticles is at least 0.5 pmol per cell of a PFC nanoparticle comprising a perfluoro-15-crown-5-ether core component. Methods of quantifying PFC nanoparticles in a sample are described in both the specific examples cited herein as well as in certain references (Morawski et al., Magnetic Resonance in Medicine 52:1255-1262, 2004). For example, quantification can be accomplished by performing magnetic resonance spectroscopy on an external standard containing a mixture of CE and PFOB emulsion in known amounts, where the ratio was determined between the CE peak and PFOB peak (˜10 ppm) areas with similar T₁ and T₂ values. This ratio is then used to calculate both the PFOB and CE content of labeled cells by analyzing magnetic resonance spectra obtained from the labeled cells. For example, magnetic resonance spectra from a PFOB labeled cell and the known quantity of the CE standard can be gathered, and the ratio between the PFOB and CE standards then used to calculate the PFOB content of the labeled cell. Conversely, magnetic resonance spectra from a cCE labeled cell and the known quantity of the PFOB standard can be gathered, and the ratio between the PFOB and CE standards then used to calculate the PFOB content of the labeled cell.

Alternatively, an external standard of NMR-grade trichlorofluoromethane (CFC-11; Sigma Chemical Co.) can be affixed to the magnetic resonance coil during all spectroscopic experiments. Fluorine spectra can then be acquired from selected volumes of undiluted crown ether nanoparticles (for example 0, 1.5, 2, 4, and 10 uL of an undiluted CE standard) and PFOB nanoparticles (0, 1.5, 2, and 4 uL) to generate a calibration curve for fluorine quantification. After applying 30 Hz line broadening, the largest PFOB peak and the single crown ether peak can be integrated with respect to the CFC-11 peak using Nuts NMR (Acorn NMR, Inc., Livermore, Calif., USA). The integrated values can then be plotted against the amount of perfluorocarbon in each sample and fit using linear regression. If necessary, corrections can be applied to the integrated values to compensate for partial-saturation effects (Fan X, et al., Int J Radiat Oncol Biol Phys 2002; 54:1202-1209). Fluorine spectra from each cell sample can then be acquired and integrated in an identical fashion. The integrated 19F signal and the calibration curves are then used to calculate the number of CE or PFOB particles in each sample.

In practicing the methods of this invention perfluorocarbon nanoparticles used are made by mixing the PFC nanoparticles in an emulsion comprising the PFC nanoparticles, various lipids, surfactants, glycerin and water. In instances where it is desirable, the PFC nanoparticles may be made fluorescent by incorporation of fluorescent-conjugated phospholipids such as 1,2-dioleoyl-sn-glycero-3-phosphoethanolamine-N-(7-nitro-2-1,3-benzoxadiazol-4-yl, or rhodamine (1,2-dioleoyl-sn-glycero-3-phosphoethanolamine-N-(lissamine rhodamine B sulfonyl). A wide variety of different lipids and surfactants may be used to coat the PFC nanoparticles. Particularly preferred are the methods and emulsions for PFC nanoparticles coating disclosed by in Lanza, G. M. et al. Circulation 94: 3334-3340, 1996. Briefly, the preferred emulsion comprises perfluorodichlorooctane (40% vol/vol, PFDCO, Minnesota Manufacturing and Mining), safflower oil (2.0%, wt/vol), a surfactant comixture (2.0%, wt/vol), 1 mol % phosphatidylethanolamine and glycerin (1.7%, wt/vol). If desired, 1 mol % N-(6-(biotinoyl)amino)hexanoyl)-dipalmitoyl-L-phosphatidyl ethanolamine may also be employed in the lipid mixture. The surfactant co-mixture includes 64 mol % lecithin, and 35 mol % cholesterol. This surfactant co-mixture is dissolved in chloroform, evaporated under reduced pressure, dried in a 50° C. vacuum oven overnight, and dispersed into water by sonication, resulting in a liposome suspension. The liposome suspension was transferred into a blender cup (Dynamics Corp of America) with perfluorodichlorooctane, safflower oil, and distilled, deionized water and emulsified for 30 to 60 seconds. The emulsified mixture was transferred to an S110 Microfluidics emulsifier and continuously processed at 10 000 PSI for 3 minutes. However, it is also contemplated that a variety of equivalent methods can be used to obtain PFC nanoparticles with coatings suitable for use in this invention. For example, it is also anticipated that the PFC nanoparticles may be mixed in an emulsion containing water, lethicin and safflower oil as described by Ahrens et al (Ibid). The perfluorocarbon nanoparticles useful in the practice of this method typically have an average diameter in a range of about 200 nm to about 300 nm.

A fundamental difference between the nanoparticle emulsions disclosed herein and previously disclosed emulsions is that the emulsions disclosed herein do not employ any type of ligand to target the nanoparticle to the cells. In this respect, the labeling method described in the instant application is a non-targeted labeling method. However, it is also believed that any of several FC or PFC nanoparticle emulsions that have been used in conjunction with various targeting ligands such as antibodies, viruses, chemotherapeutic agents, receptor agonists and antagonists, antibody fragments, lectins, albumins, peptides, hormones, amino sugars, lipids, fatty acids, nucleic acids, and cells may be used in the practice of the present invention by simply omitting the ligand from the nanoparticle emulsion. Examples of such nanoparticle emulsions that contain targeting ligands are disclosed in the following U.S. patents and U.S. published patent applications: U.S. Pat. Nos. 5,690,907, 5,780,010, 5,958,371, 6,548,046, 6,676,963, 2003/0086867A1, 2004/0058951A1, and 2004/0248856A1, the entire disclosures of each of which are incorporated herein by reference. It is anticipated that the emulsions taught in U.S. patents and U.S. published patent applications U.S. Pat. Nos. 5,690,907, 5,780,010, 5,958,371, 6,548,046, 6,676,963, 2003/0086867A1, 2004/0058951A1, and 2004/0248856A1, could be used in the present invention by simply omitting the ligand from the nanoparticle emulsion taught in these patents and patent applications.

### Methods of Imaging Cells at a Magnetic Field Strength of 1.5 T

The instant invention further provides for methods obtaining a magnetic resonance image of a plurality of cells introduced into a subject at a magnetic field strength of 1.5 T. In practicing this method, it is first necessary to obtain a plurality of cells with an intracellular perfluoro-15-crown-5-ether nanoparticle concentration of at least 0.5 pmol per cell. A variety of methods can be used to obtain cells with an intracellular concentration of perfluoro-15-crown-5-ether nanoparticle concentration of at least 0.5 pmol per cell. Cells with the required PFC nanoparticle concentrations can be obtained through the use of electroporation, transfection, ultrasound or sonication based techniques.

Transfection based delivery of the PFC nanoparticles is typically achieved by use of cationic transfection reagents. For example, Ahrens et al. (ibid) have described introduction of PFC nanoparticles into cells through use of Lipofectamine™ (Invitrogen, Carlsbad, Calif.). In brief, suitable amounts of the cationic transfection reagent, lipid-emulsion coated PFC nanoparticles, and cell growth media are pre-mixed and briefly incubated. The mixture of the cationic transfection reagent, PFC nanoparticles and cell growth media are then incubated with the cells for a suitable period of time and at a suitable temperature. In Ahrens et al., incubation was for 3 hours at 37° C. under 5% carbon dioxide for one cell type (fetal-skin derived dendritic cells). Following this treatment, the transfection reagent and uninternalized PFC nanoparticles are removed by washing. Other suitable cationic transfection reagents include TransFectin™ (BioRad, Inc., Hercules, Calif., USA), BD CLONfectin™ (TAKARA BIO, Inc., Mountain View, Calif.), and FuGENE® transfection Reagent (Roche Diagnostics, Indianapolis, Ind., USA).

Ultrasound and sonication are two additional techniques for obtaining cells with an intracellular perfluoro-15-crown-5-ether nanoparticle concentration of at least 0.5 pmol per cell. Such techniques are described in detail for both targeted and non-targeted PFC nanoparticles (Crowder et al. Ultrasound Med Biol. 2005 December; 31(12):1693-700). In brief, the perfluorocarbon nanoparticles are prepared essentially as previously described herein and as in Lanza et al, 1996. Targeted molecules can be obtained by incorporating a variety of ligands into the surface of the PFC nanoparticle. For example, a thiolated peptidomimetic vitronectin antagonist of the αᵥβ₃-integrin (Bristol-Myers Squibb Medical Imaging, Inc., Billerica, Mass., USA) can be covalently coupled to MPB-PEG2000-phosphatidylethanolamine and combined with other surfactant components. Ultrasound is applied to the cells in culture with a clinical medical imager (Acuson Sequoia; Siemens, Malvern, Pa., USA) with a broadband (2 to 3.5 MHz, 3V2a) phased-array transducer, which is applied from the side at a 30° angle. A tissue culture dish is modified by drilling a hole into the dish and using a watertight sealant to secure a coverslip to the bottom of the dish. A 2% agarose disk, which couples the ultrasound to the cells, is made to fit the dish and a hole is cored out over the coverslip. The experiments can take place on top of an inverted phase-contrast microscope which permits simultaneous microscopic visualization of cell interactions during exposure to calibrated levels of ultrasound energy (i.e., mechanical index MI: for example, a machine power output setting: 1.9; frequency: 2 MHz; focal zone setting: 60 mm; exposure time: 5 min). Ultrasound settings can be chosen for other devices such that nanoparticles experienced the highest visually apparent radiation force. Cells are grown on the coverslip at 37° C. to confluence before exposure to the experimental conditions described above.

In practicing this method of imaging cells introduced into a subject at clinical field strengths of 1.5 T, a variety of magnetic resonance imaging methods may be employed. For example, the magnetic resonance imaging method may comprise a steady state free precession pulse sequence (SSFP), a balanced-fast field echo imaging sequence or a combination of SSFP and a fast field echo imaging sequence. The balanced fast field echo imaging sequence may comprise an echo time (TE) of 5 ms, a time to repetition (TR) of 10 ms, 512 signal averages, a 2.5×2.5 mm reconstructed in-plane resolution, a 60 degree flip angle, a 35 mm slice thickness, and a total scan time of between 2 to 10 minutes. Parameters for determining the scan time include the degree of PFC labeled cell localization (i.e., the volume or area that the cells occupy in the subject) as well as the intracellular concentration. When the CE nanoparticles-containing cells are tightly localized within the subject or when the CE nanoparticles are at a high intracellular concentration (i.e., greater than 0.5 pmol/cell), scan times of 2-5 minutes may be sufficient to acquire the magnetic resonance imaging data. However, when the cells are less closely localized within the subject or when the CE nanoparticles are at concentrations of about 0.5 pmol/cell, longer scan times of 5-10 minutes may be required. In a non-limiting example shown herein, cells with an intracellular CE concentration of about 0.5 pmol/cell were introduced into a subject and imaged by using a scan time of 7 minutes.

In certain examples described herein, ¹⁹F imaging is enabled in a clinical 1.5 T Philips (Phillips Medical Systems, Bothell, Wash., USA) Magnetic Resonance (MR) scanner by modifying the system to include a specialized channel tuned for fluorine nuclei and a series of surface and volume RF coils tuned to the same frequency (60.1 MHz). However, for MR scanners that have fluorine channels and appropriate associated coils, the specialized channel and coils are not needed. The inventors herein believe that any of a number of known tuned coils for fluorine imaging can be used in the practice of the present invention.

Methods of Obtaining Two Distinct Magnetic Resonance Imaging or Spectroscopy Data Sets Derived from Two Distinct Cells Introduced into a System

It is recognized that in certain instances it may be useful to introduce into a system two distinct cells and independently track the fate of the cells over the course of time. For example, certain therapeutic regimens may call for the introduction of more than one cell type into a system that is a subject. For purposes of illustration, it may be advantageous in certain instances to simultaneously introduce into a subject both an endothelial precursor cell and a dendritic cell. Using the methods taught herein, each cell type could be labeled with a different PFC nanoparticle (i.e., the endothelial precursor cell could be labeled with a CE nanoparticle while the dendritic cell could be labeled with a PFOB nanoparticle or vice versa), introduced into the subject, and MR imaging or spectroscopy data specifically collected from each cell type. MR imaging or spectroscopy data collected for the CE nanoparticle would thus yield information on the fate of one set of cells whereas MR imaging or spectroscopy data collected for the PFOB nanoparticle would thus yield information on the fate of the other set of cells.

As the foregoing discussion illustrates, distinct cell sets may be labeled with either CE or PFOB in any temporal order. Moreover, the cells may be labeled with either CE or a PFOB nanoparticles by electroporation, transfection, ultrasound, and sonication. Alternatively or additionally, the cells that are endothelial precursor cells may be labeled by incubating the cells in a cell culture media containing a plurality of perfluorocarbon nanoparticles for a period of time and at a perfluorocarbon nanoparticle concentration sufficient to result in internalization of a detectable level of perfluorocarbon nanoparticles as described in the instant invention.

Distinct MR imaging and spectroscopy data sets from cells labeled with CE or PFOB nanoparticles may be obtained from a variety of different types of systems. The systems may be in vitro systems (i.e., systems where cells are propagated outside of an organism). Non-limiting examples of in vitro systems include systems for regenerating a tissue or an organ outside of a host organism. Non-limiting examples of regenerating tissue types may include bone, cartilage, vascular, pancreatic, liver, heart, lung, bladder, muscle and neural tissue. Alternatively or additionally, the in vitro system may be a test tube, a petri dish, a microtiter plate well, a roller bottle, and a cell culture reactor.

Distinct MR imaging and spectroscopy data sets from cells labeled with CE or PFOB nanoparticles may be also obtained from a system such as a living organism. This living organism may be a mammal. Non-limiting of mammalian systems that could be used include a mouse, a rat, a rabbit, a cat, a dog, a pig, a cow, a horse, a monkey, or a human.

It is envisioned that distinct MR imaging and spectroscopy data sets from cells labeled with CE or PFOB nanoparticles may be also obtained through use of a magnetic field strength of 11.7 T. Alternatively or additionally, a magnetic field strength of 1.5 T may be employed to gather the MR spectroscopy of imaging data sets.

Magnetic resonance imaging methods that specifically detect a 19F MRI signal from either a PFOB or CE nanoparticle core component fundamentally comprise methods where an excitation signal for one type of nanoparticle (either CE or PFOB) is used that does not overlap with the resonance frequency for the other nanoparticle. For example, specific detection of a perfluoro-15-crown-5-ether core component labeled cells comprises use of an excitation signal centered at a frequency that is substantially the same as the resonance frequency of the perfluoro-15-crown-5-ether core component, where that excitation signal bandwidth does not overlap any of several resonance frequencies of the perfluorooctylbromide core component. While the excitation signal is preferably centered on an excitation frequency or frequencies of a given type of nanoparticle, the key feature is that the excitation frequency not overlap any resonance frequencies of a nanoparticle that is to be excluded from the data set. To accomplish acquisition of data from resonance frequencies for only one type of a nanoparticle, the excitation signal is typically a narrow bandwidth excitation signal. In the example cited above for specifically detecting CE labeled cells, the narrow bandwidth excitation signal may have a bandwidth of 1002 Hz. Specific detection of a 19F MRI signal from a perfluorooctylbromide core component comprises use of an excitation signal centered at a frequency that is substantially the same as the resonance frequency of at least one selected spectral peak generated by the perfluorooctylbromide core component, where the excitation signal bandwidth does not overlap the resonance frequency of said perfluoro-15-crown-5-ether core component. This excitation signal is typically a narrow bandwidth excitation signal. For example, PFOB labeled cells can be specifically detected by a narrow bandwidth excitation signal with a bandwidth of 900 Hz for a single resonance peak for said perfluorooctylbromide core component that is 600 Hz removed from a perfluoro-15-crown-5-ether resonance peak. Alternatively, PFOB labeled cells can be specifically detected by a narrow bandwidth excitation signal with a bandwidth of 2018 Hz for a plurality of resonance peaks for said perfluorooctylbromide core component that are 2000 Hz removed from a perfluoro-15-crown-5-ether resonance peak.

The methods described herein are also suitable for obtaining magnetic resonance spectroscopy data by specifically detecting a 19F MRI signal from either a PFOB or CE nanoparticle core. These methods may comprise acquisition of volume selective spectra by image-selective in vivo spectroscopy.

### EXAMPLES

### Example 1

### Labeling Stem/Progenitor Cells with PFC Nanoparticles

Liquid PFC nanoparticles were formulated using methods previously developed in our laboratories (Lanza, G. M. et al. Circulation 94: 3334-3340, 1996). Briefly, the emulsions comprised 20% (v/v) perfluorocarbon (PFC) such as either perfluorooctylbromide (PFOB) or 15-crown-5 ether (CE), 1.5% (w/v) of a surfactant/lipid co-mixture, and 1.7% (w/v) glycerin in distilled, deionized water. Fluorescent nanoparticles contained fluorescent-conjugated phospholipids of either 2.05 mole % of NBD (1,2-dioleoyl-sn-glycero-3-phosphoethanolamine-N-(7-nitro-2-1,3-benzoxadiazol-4-yl)) or 0.135 mole % of rhodamine (1,2-dioleoyl-sn-glycero-3-phosphoethanolamine-N-(lissamine rhodamine B sulfonyl)) (Avanti Polar Lipids, Inc., Alabaster, Ala.) in the surfactant layer. The mixture of surfactant components, PFC and water was blended and then emulsified at 20,000 PSI for four minutes in an ice bath with an estimated temperature range of about 0° C. to 4° C. (S110 Microfluidics emulsifier, Microfluidics, Newton, Mass.). Particle size analysis by laser light scattering (Brookhaven Instruments Corp., Holtsville, N.Y.) measured sizes of 224 nm and 233 nm for PFOB and CE formulations, respectively.

Mononuclear cells (MNCs) were isolated by density gradient centrifugation with Ficoll-Paque Plus (Amersham Biosciences, Piscataway, N.J.) from human umbilical cord blood obtained from the Cardinal Glennon Children's Hospital (St. Louis, Mo.). MNCs were plated at concentrations of 5×10⁵ cells/cm² on fibronectin-coated plates (RetroNectin; Takara, Otsu, Japan) and grown in modified endothelial growth media (Clonetics EGM™-2-Endothelial Cell Medium-2; Cambrex, East Rutherford, N.J.) adjusted to a final concentration 20% FBS (Fetal Bovine Serum). This modified endothelial growth media is designed to promote differentiation along the endothelial lineage. At day 2, non-adherent cells were removed and transferred to fresh fibronectin-coated plates. After 7-14 days, cells were incubated for 12 hours with a 30 pM concentration of fluorescent perfluorocarbon nanoparticles containing either a perfluorooctyl bromide (PFOB) or 15-crown-5-ether (CE) core component with NBD or rhodamine-tagged phosphatidylethanolamine lipids, respectively. In certain experiments aimed at determining the contribution of free or non-internalized nanoparticles to the fluorine signal, cells were briefly incubated for only 10 minutes at the 30 pM nanoparticle concentration to allow introduction of a background signal in the absence of nanoparticle internalization. After incubation for the indicated time period (i.e., 12 hours for internalization or 10 minutes for non-internalized controls), cells were prepared by removing free nanoparticles with PBS washing, detaching adherent cells from the surface of the fibronectin-coated plates with cell dissociation solution (Sigma, St. Louis, Mo.) and collecting the cells by centrifugation.

### Example 2

### Confocal Microscopic Imaging and Immuno-Characterization of PFC-Labeled Cells

To image PFC-labeled cells with confocal microscopy, labeled cells treated as described in Example 1 were first subjected to 2% paraformaldehyde fixation for 30 minutes. For confocal imaging fixed cells were placed in 1.5 glass bottom culture dishes (Bioptechs Inc., Butler, Pa.). Fluorescence imaging of cell sections was conducted with a confocal microscope (Zeiss Meta 510, Thornwood, N.Y.), using standard filter sets. The location of nanoparticles with respect to the cell was determined with simultaneous differential interference contrast (DIC) imaging.

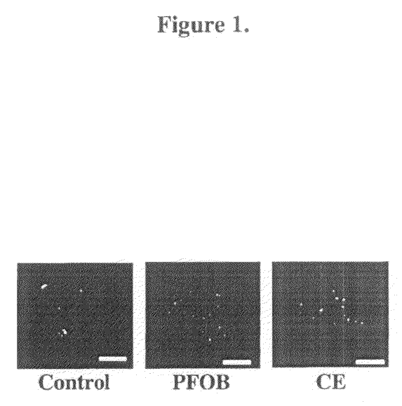

Internalization of nanoparticles occurred without aid of any additional transfection agents or methods, characterized by abundant uptake and distribution throughout the cytosol for both PFOB and CE nanoparticles. Qualitatively for individual cells that internalized nanoparticles, a greater uptake of nanoparticles appeared for PFOB-loaded as compared with CE-loaded cells (FIG. 1). Cell sectioning revealed that nanoparticles were not within the nucleus. Quantitative analysis of cell loading by flow cytometry showed 77.4+/−2.6 percent of the cell population contained nanoparticles for both types of particles.

To characterize the cell population derived from the combined influence of the conditioning media and elapsed time, we probed cells with monoclonal antibodies for characteristic stem and progenitor cell markers and analyzed expression with flow cytometry. The cell culture conditions described in Example 1 are expected to generate endothelial progenitor cells (EPCs) from the mononuclear cells isolated from umbilical cord blood. Cells were removed with cell dissociation solution (Sigma, St. Louis, Mo.) both before and after incubation with fluorescently-labeled nanoparticles, and analyzed for cell surface markers with an array of monoclonal antibodies including CD34-PE (phycoerythrin)-Cy7, human CD45-APC (allophycocyanin), human CD31-PE, Tie-2-APC (Becton Dickinson, San Jose, Calif.), or CD133-PE (Miltenyi Biotechnology, Bergish, Gladbach, Germany). Immuno-stained samples were analyzed on a Coulter FC-500 flow cytometer with RXP analysis software (Beckman-Coulter, Miami, Fla.).

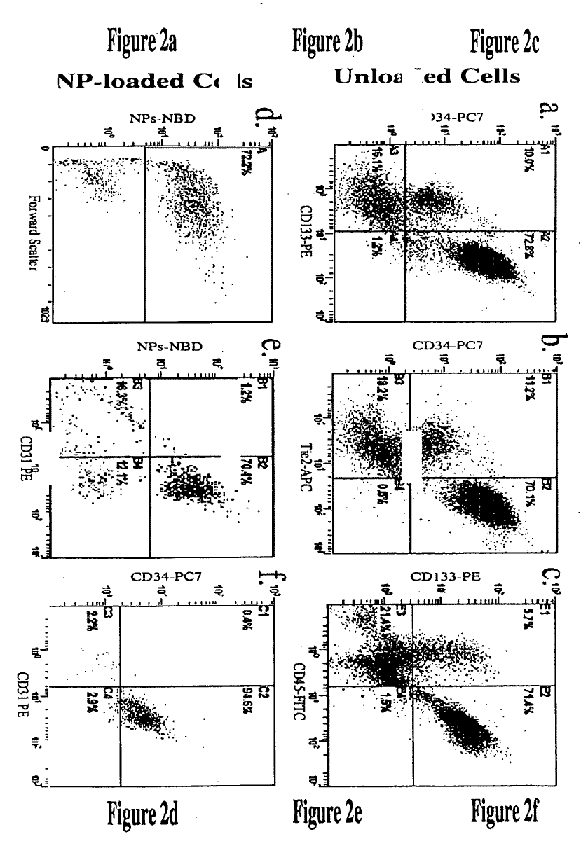

A significant part of the population (˜70%) co-expressed the hematopoietic marker CD34 and the progenitor marker CD133, and a smaller population (10%) were CD34⁺CD133⁻ (FIG. 2a). Co-expression of CD34 and the endothelial marker Tie-2 occurred in 70% of the cells whereas a smaller population (˜11%) of the cells were CD34⁺Tie-2⁻ (FIG. 2b). Probing for the hematopoietic/leukocyte marker CD45 revealed a small portion (˜6%) of CD133⁺CD45⁻ cells (FIG. 2c). Approximately 72% of all cells contained nanoparticles (FIG. 2d). Almost all cells loaded with nanoparticles expressed the endothelial marker CD31, but a small portion (˜12%) of CD31⁺ cells remained unlabeled (FIG. 2e). We observed that almost 95% of the loaded cell population expressed both CD34 and CD31 (FIG. 2f).

In general, flow cytometry revealed ≧68% of the entire cell population contained nanoparticles and stained positive for CD34, CD133, and CD31 markers. CD34, CD133, CD31, and Tie-2 are characteristic markers of Endothelial Precursor Cells (EPCs). However, CD45+, which is typically down regulated in EPCs, was manifest in ˜70% of the overall population of cells. Adjustment of the culture conditions or more prolonged exposure to culture media may be sufficient to obtain a lower percentage of CD45+ cells.

### Example 3

### Effects of PFC Nanoparticle Labeling on Cell Viability and Function

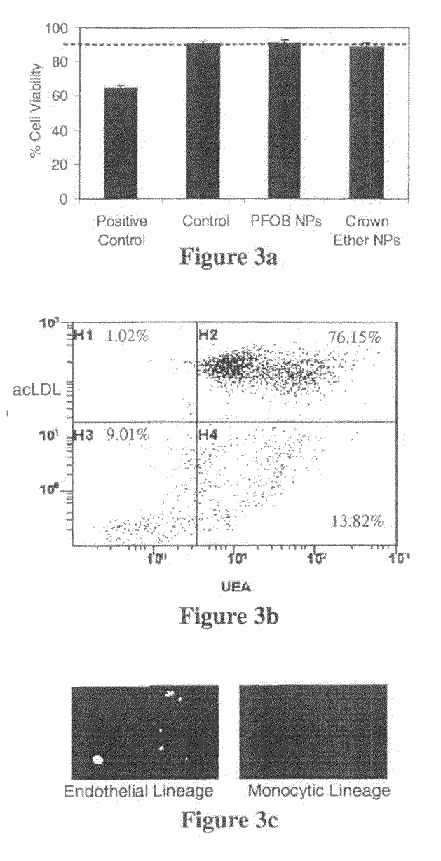

To determine if PFC nanoparticle labeling affected cell viability, the percent (%) cell survivability after labeling of cells as described in Example 1 was determined by trypan blue exclusion. Cells labeled with either CE or PFOB nanoparticles were removed with trypsin, resuspended in PBS, and diluted 1:1 with 0.4% trypan blue (Sigma, St. Louis, Mo.). For a positive control, cells were heated at 45° C. for 15 min. The number of viable and nonviable cells was counted using a hemocytometer with the percentage of trypan blue positive cells was used to calculate cell survival. We found high cell survivability (˜90%) of cells subjected to labeling procedure of Example 1 with no significant difference from non-labeled control cells (FIG. 3a). Positive control cells exposed to high temperature manifested a substantial loss of cell viability (˜60% cell survivability). Accordingly, neither PFOB nor CE nanoparticles exerted any untoward effect on cell viability.

To evaluate cell function after nanoparticle loading, cells were incubated for 12 hours with non-fluorescent nanoparticles and subsequently incubated with DiI-acetylated-LDL, which is endocytosed independently through the cell's surface scavenger receptors. Internalization of acLDL is a functional characteristic of both monocytic and Endothelial Precursor Cells while the presence of fucose is a compositional characteristic of Endothelial Precursor Cells but not monocytic cells. PFC labeled cells obtained as described in Example 1 were assayed for acLDL uptake and the presence of fucose by incubating the cells with FITC-labeled UEA-1, a lectin that binds the sugar fucose, to differentiate between Endothelial Precursor Cells (double positive acLDL uptake +/fucose+) versus monocytic (single positive acLDL+) cells. This was accomplished by incubating the labeled cells with acetylated LDL at 15 mg/mL for 4 hrs and staining after fixation with Ulex europaeus agglutinin (UEA)-1 at 10 mg/mL for 1 hr. Analysis of function with flow cytometry revealed a large population of cells (˜76%) that could bind and internalize acLDL (FIG. 3b), despite being loaded previously with nanoparticles. In addition, acLDL+ cells stained positive for UEA-1, indicating that the cell population exhibited functional and structural features similar to cells of endothelial lineage. These results were confirmed by confocal microscopy illustrating acLDL particles within the cytosol. Cells positive for UEA-1 manifested diffuse labeling on the surface with smaller areas of more intense localization (FIG. 3c), suggesting that fucose is heterogeneously distributed throughout the cell surface.

### Example 4

### Imaging of Labeled Cells In Vitro at 11.7 T and 1.5 T Field Strengths

For 11.7 T spectroscopy and imaging, cells were loaded with CE or PFOB nanoparticles, preserved with paraformaldehyde fixation, and condensed into a pellet by centrifugation. To quantify the amount of perfluorocarbon incorporated into the cells, known amounts of PFOB or CE nanoparticles were utilized as standards for CE or PFOB loaded cells respectively.

¹⁹F MRS/MRI of labeled cells was performed on a Varian 11.7 T scanner using a custom-designed 0.5 cm 4-turn solenoid RF coil. Labeled cells were contained within a centrifuge tube and analyzed together with an internal standard of PFOB or CE nanoparticle emulsions provided by inserting the cell tube into a slightly larger tube containing the emulsion standard. ¹⁹F MRS (TR: 1s, number of averages: 128, acquisition time: 2 minutes) of cells was performed for quantitative evaluation of intracellular labeling of nanoparticles. For quantification an external standard was made containing a mixture of CE and PFOB emulsion in known amounts, where the ratio was determined between the CE peak and PFOB peak (˜10 ppm) areas with similar T₁ and T₂ values. For cell pellets a 5 μL internal standard was provided containing either 100% PFOB or 10% CE emulsion for CE or PFOB-labeled cells, respectively. The spectra of CE and PFOB were used to define the offset frequency of RF output for ¹⁹F imaging. ¹⁹F images of PFOB and CE labeled cells were acquired using a multi-slice gradient echo sequence (TE, 3 ms, TR, 50 ms, flip angle, 20°, FOV, 1×1 nm, image resolution, 156×156 μm², slice thickness, 2 mm, number of average, 128, acquisition time, 7 minutes).

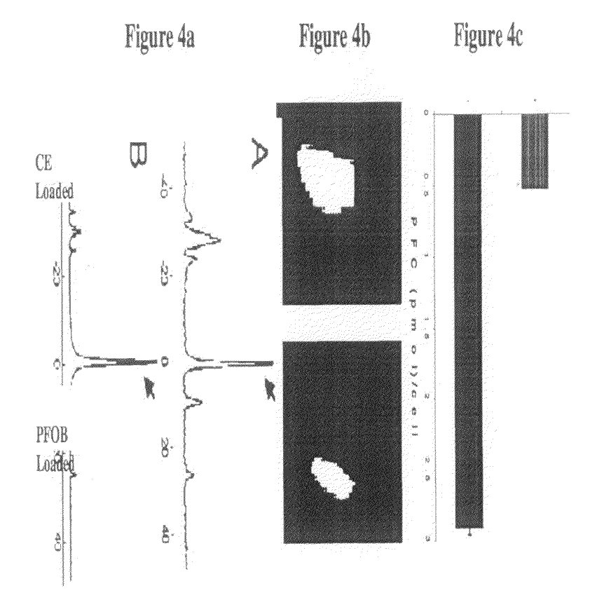

MR spectroscopy indicates that the spectrum of CE comprises a single ¹⁹F peak (FIG. 4a, arrow); whereas the spectrum of PFOB is characterized by multiple peaks that span 60 ppm in the frequency domain (FIG. 4a, all other peaks). The spectra were readily detectable (˜2 min acquisition time) with little background noise. No ¹⁹F signal was detected from control cells. For ¹⁹F MR imaging of PFOB labeled cells, the center frequency of the second PFOB peak (˜10 ppm) from the right was selected as the RF output frequency; whereas CE imaging was performed on the single peak. Both CE and PFOB loaded cell pellets were rapidly (7 min scan time) imaged in vitro revealing a stronger native signal originating from the CE cells, consistent with the spectral energy being concentrated within a single fluorine peak instead of being distributed between several as was the case for the similar concentration of PFOB. Although the signal intensity of PFOB labeled cells was lower than that of CE labeled cells, ¹⁹F MRI shows that PFOB labeled cells are detectable even at high image resolution. Quantification of the concentration of perfluorocarbon in the cell pellets after 12 hours of incubation indicated that we achieved concentrations of up to 2.96 pmol of perfluorooctylbromide fluorocarbon (PFOB) per cell and of up to about 0.53 pmol of perfluoro-15-crown-5-ether (CE) per cell (Table I). Quantification was accomplished by the higher concentration of PFOB versus CE nanoparticles, which was corroborated with confocal micrograph images (FIG. 1).

TABLE I
Quantification of Internalized PFC nanoparticles

| | |
|---|---|
| Perfluorooctylbromide/cell | Perfluoro-15-crown-5-ether/cell |
| (pmol/cell) | (pmol/cell) |

| | | | |
|---|---|---|---|
| Replicate 1 | 2.95 | replicate 1 | 0.51 |
| Replicate 2 | 2.96 | replicate 2 | 0.53 |
| Replicate 3 | 2.81 | replicate 3 | 0.50 |
| Average | 2.90 | Average | 0.51 |
| SD | 0.082 | SD | 0.015 |
| SEM | 0.048 | SEM | 0.009 |

Cells labeled with Crown Ether (CE) PFC as described in Example 1 were also imaged in vitro with a 1.5 T clinical scanner. To determine the contribution of free or non-internalized nanoparticles to the fluorine signal, cells were briefly incubated (˜10 minutes) with a 30 pM nanoparticle concentration to allow introduction of background signal, but not internalization.

The fixed and pelleted cells were imaged at 1.5 T using a clinical scanner (Philips Intera CV, Philips Medical Systems; Best, Netherlands) fitted with a dedicated ¹⁹F channel and a 7-cm square surface coil tuned to 60.1 MHz for transmit and receive. ¹⁹F projection images of the cells were acquired using a balanced Fast Field Echo (FFE) sequence (TE: 5 ms, TR: 10 ms, 512 signal averages, 0.5×0.5 mm reconstructed in-plane resolution, 60 degree flip angle, 25 mm slice thickness, ˜9 min total scan time). Matching ¹H images were acquired for comparison using the quadrature body coil for transmission and 4 cm diameter surface coil for receive (multi-slice T1-weighted spin echo sequence, TE: 15 ms, TR: 500 ms, 2 signal averages, 0.5×0.5 mm reconstructed in-plane resolution, 70 degree flip angle, 5 mm slice thickness with 1 mm gap, ˜3:30 min total scan time).

Proton (¹H) imaging was used to locate the samples, but only cells incubated with PFC nanoparticles for 12 hours (and not 10 minutes) as described in Example 1 could be detected by fluorine (¹⁹F) imaging at 1.5 T. This result confirms that the fluorine signal is specific for imaging cells with internalized nanoparticles, thereby rendering the possibility of imaging non-specific accumulation less likely. For imaging times of less than 10 minutes at 1.5 T, the ¹⁹F imaging sequence generated a large signal-to-noise ratio of 21.

### Example 5

### Imaging of Labeled Cells In Situ at 11.7 T and 1.5 T Field Strengths

To determine the feasibility of detecting cells at specific tissue sites after local delivery, one million cells (2×10⁵/μL) labeled with CE nanoparticles essentially as described in Example 1 were injected into a mouse thigh skeletal muscle. For in situ imaging, an adult C57/BL6 mouse was injected in the right thigh with approximately one million labeled stem/progenitor cells diluted in 50 uL PBS. ¹H and ¹⁹F MR imaging were performed on a Varion 11.7 T Inova console using a 3 cm surface coil tuned to either ¹H or ¹⁹F frequency and a multi-slice gradient echo sequence (¹⁹F parameters—image matrix: 64×64, FOV 3×3 mm, slice thickness: 2 mm, TR: 50 ms, TE: 3 ms, number of averages 128, ˜7 minute total acquisition time; ¹H parameters—image matrix: 256×256, FOV 3×3 mm; slice thickness 2 mm, TR: 300 ms, TE: 3 ms, number of average 2, ˜2.5 min acquisition time).

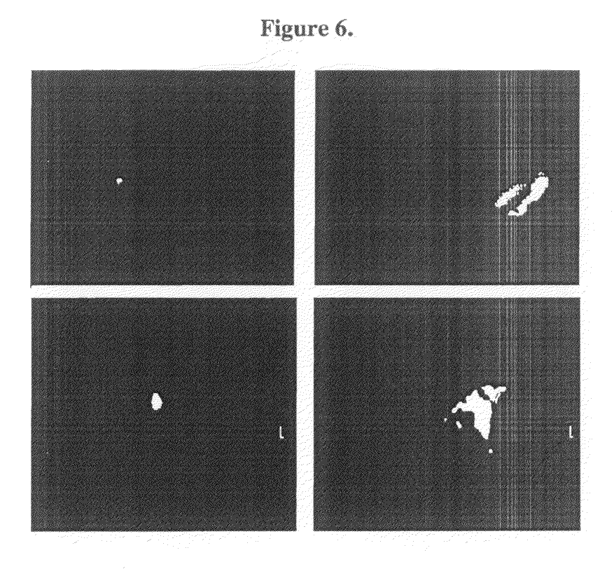

At 11.7 T, the cells were imaged rapidly in approximately 7 minutes by tuning to the CE peak (FIG. 6a). Overlaying the fluorine image atop a conventional proton image reveals that the cells are located near the mouse femur and adjacent to an air bubble introduced during injection to help verify the location of the ¹⁹F signal (FIG. 6b).

To capture images of cells introduced into a mouse at 1.5 T, four million cells were fixed and labeled essentially as described in Example 1. The cells were combined and resuspended in 50 micro liters of Phosphate Buffered Saline (PBS) to generate a final concentration of approximately 80,000 cells per micro liter. They were then injected into the right hind limb of a euthanized mouse and imaged at 1.5 T using a clinical scanner (Philips Intera CV, Philips Medical Systems; Best, Netherlands) fitted with a dedicated 19F channel. A 5-cm square surface coil tuned to 60.1 MHz for transmit and receive was used to generate a projection image of the cells with a 2D balanced Fast Field Echo (FFE) imaging sequence (TE 5 ms, TR 10 ms, 512 signal averages, 2.5×2.5 mm reconstructed in-plane resolution, 60 degree flip angle, 35 mm slice thickness, 7 min total scan time). ¹H images were acquired for comparison using the quadrature body coil for transmission and 4 cm diameter surface coil for receipt (3D T1-weighted turbo spin echo sequence, TE 15 ms, TR 363 ms, 7 signal averages, 0.2×0.2 mm reconstructed in-plane resolution, 90 degree flip angle, 2 mm slice thickness, 10 slices, ˜11 min total scan time).

In the in situ images obtained at 1.5 T, a similar injection of approximately 4 million CE nanoparticle labeled cells (8×10⁴/μL) produced a strong fluorine signal (FIG. 6c) in approximately 7 minutes. When overlayed atop a proton image, the fluorine signal is located at the mouse thigh just posterior to the bright signal from the gut (FIG. 6d). These images suggest that the level of nanoparticle uptake by cells was high enough that upon dilution and injection into a mouse leg, sufficient ¹⁹F is present to allow image acquisition at either 11.7 T and 1.5 T in 7 minutes. In addition, all images were acquired with essentially no background signal to enable definitive assignment of the fluorine signal to the labeled cells, rendering this a viable technique for imaging stem cells quickly in vivo on a clinical imager.

### Example 6

### Spectroscopic Analysis of Fibrin-Targeted Blood Clots

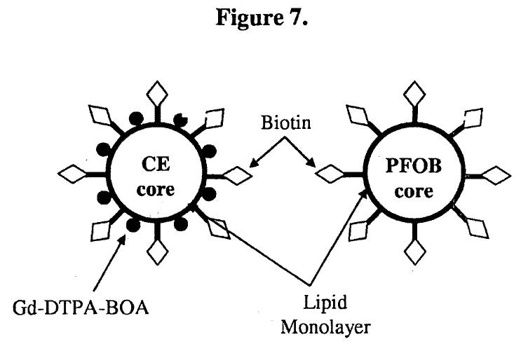

Fresh-frozen canine plasma, anticoagulated with sodium citrate, was used to form fibrin clots by combining plasma, 100 mM calcium chloride (3:1 v/v), and 5 U thrombin (Dade Behring, Germany) on 2 cm diameter nitrocellulose discs and distributed into a 12-well cluster plate. The clots were serially incubated with 125 μg biotinylated anti-fibrin monoclonal antibodies (1H10; Edgell T, et al. Thrombosis & Haemostasis. 1996; 75(4):595-599; Raut S, et al. Thrombosis & Haemostasis. 1996; 76(1):56-64) overnight at 4° C., followed by 125 μg avidin for 1 hour at 37° C., and then 100 μl of the selected biotinylated nanoparticle mixture for 1 hour at 37° C. to complete the binding. A schematic view of the PFC nanoparticles used is shown in FIG. 7. The relative concentration of ¹⁹F signal-generating nanoparticles bound to the clots was varied by admixing volumes of CE- and PFOB-based emulsions in ratios of 1:0, 2:1, 1:2, and 0:1, respectively, and by applying each combination to three individual clots. The treatments and replicates were aligned in the 12-well plate such that each of the four columns contained 3 replicates of the same treatment (for image acquisition purposes). All samples were rinsed three times with sterile saline after each incubation step to remove unbound reactants.

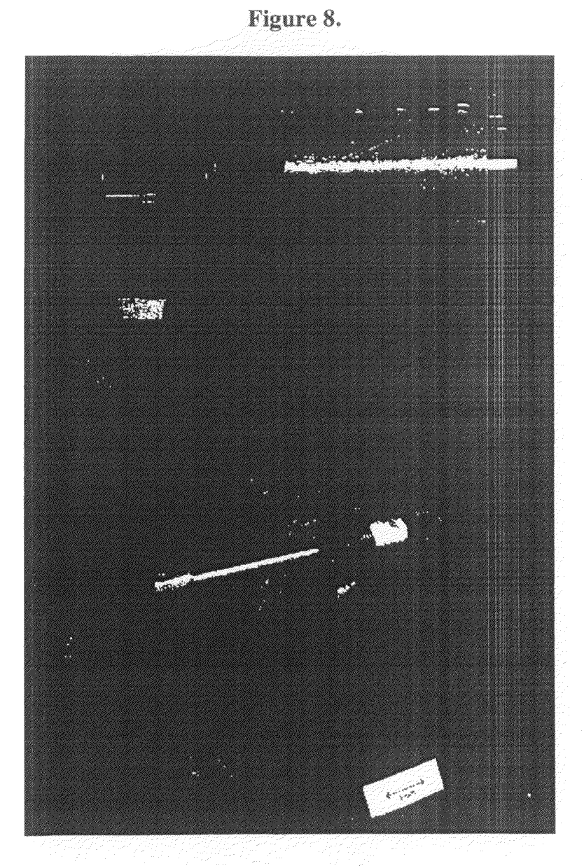

All imaging and spectroscopy was performed on a 1.5 T clinical MR system (NT Intera, Philips Medical Systems, Andover, Mass.) with peak gradients of 30 mT/m (150 mT/m/ms) and outfitted with a secondary radio frequency (RF) transmit/receive system tuned for ¹⁹F. Proton images were acquired using the standard built-in quadrature body coil for RF transmission and either the built-in body coil or a quadrature birdcage (head) coil for reception. All fluorine spectra and images were collected using an “in-house” 14 cm diameter×14.5 cm long volume coil in both transmit and receive modes. The functional operation and advantages of this single turn solenoid (STS) coil, also known as a loop-gap resonator, have been discussed in the literature (Hornak J P, et al Magn Reson Imaging. 1987; 5(3):233-237). The coil was constructed by adhering a copper sheet onto a cast Acrylic cylinder. This apparatus is shown in FIG. 8. The tuning capacitance was distributed along the coil length in the form of high-power chip capacitors (American Technical Ceramics, Huntington Station, N.Y.), and a high-power variable tuning capacitor (Polyflon, Norwalk, Conn.). The load was inductively coupled via a coupling loop attached to a flexible cylindrical header, which provided a well-behaved load coupling adjustment without the use of any elaborate mechanical drive mechanism. Initial loading and matching adjustments were performed, with samples in place, using a network analyzer (HP8751A, Agilent, Palo Alto, Calif.), and further adjustments, as needed, were made in the MR scanner.

Survey (proton) images were acquired to localize the 12-well plate in the scanner. To characterize the clots and measure relaxation times, T1-weighted gradient echo images (TE/TR=2.36/110 ms, flip=80°, FOV=L60×200 mm, matrix=269×336, reconstructed at 512², slice thickness=5 mm, 16 averages, scan time=6.3 min) were acquired (using the birdcage coil) perpendicular to the disc of the clot and oriented such that all four groups would be visible in a single imaging slice. In the same orientation, from which to calculate T1, a single slice inversion recovery sequence (i.e., the Look-Locker technique; Look D C, Locker D R. Rev Sci Instrum. 1970; 41(2):621-627) was performed with multiple echoes sampled every 78 ms by 15° flip angle excitations (TR=2s (between IR pulses), TE=2 ms, 20 echoes, FOV=109×180, matrix=166×192 zero padded to 256², slice thickness=5 mm, 10 averages, scan time=3.0 min). This was repeated three times, one imaging slice for each clot replicate.

Fluorine spectroscopy was performed both with and without volume selection techniques. To begin, non-selective free induction decay (FID) sampling was performed. With the center frequency chosen to be that of CE and a spectral bandwidth of 8000 Hz, FID data were acquired (4096 samples, TR=4000 ms, flip angle=90 (block pulse), 16 averages, scan time=64s). Four volume selective acquisitions, using image-selective in vivo spectroscopy (ISIS; Ordidge R J, et al., Magnetic Resonance in Medicine. 1988; 8(3):323-331; Keevil S F, et al., NMR in Biomedicine. 1992; 5(4):200-208), were acquired and positioned such that the three repetitions of each of the four groups were sampled as a 60 cm³ volume (TR=5000 ms, bandwidth=8000 Hz, 4096 samples, 472 averages, scan time=39 min). For similar comparison, this high Signal to Noise Ratio (SNR) acquisition, which was much longer than necessary, was repeated with all four groups combined.

Imaging of the ¹⁹F nuclei utilized a variety of techniques to detect and discriminate perfluorocarbon resonant peaks. Multislice (TE/=1.6/3.8 ms, flip=90°, FOV=256×320 mm, matrix=102×128, reconstructed at 1282, receive BW=1460 Hz/pixel, slices=3, thickness=2 cm, gap=0.5 cm, 512 averages, scan time=8.5 min) or single thick slice (TE/TR=1.95/3.90 ms, flip=90°, FOV=208×256 mm, matrix=102×128, reconstructed at 128², receive BW=681 Hz/pixel, slice thickness=9 cm, 512 averages, scan time=2.8 min) acquisitions were oriented perpendicular to the clots and employed a true steady-state free precession (aka, “balanced” FFE) pulse sequence, which, unlike spoiled gradient echo techniques, allows very short TRs since the steady-state transverse magnetization is dependent on flip angle and not TR (where TR<<T1; Haake E M, et al. in. Magnetic Resonance Imaging Physical Principles and Sequence Design. New York: John Wiley & Sons; 1999; Perrin R L, et al., J Magn Reson Imaging. December 2004; 20(6):1030-1038; Reeder S B, et al., Magn Reson Med. July 2004; 52(1):123-130). The frequency encoding direction was perpendicular to the plane of the clots to avoid potential signal overlap from neighboring clots. Initial images were acquired with a wide excitation bandwidth (2018 Hz) centered near the CE peak and including the neighboring PFOB peak in the signal. Narrow bandwidth selective acquisitions were performed to visually discriminate PFC nanoparticle species. To select the CE nanoparticles, the center frequency was chosen to be that of the single CE peak with an excitation bandwidth of 1002 Hz. The PFOB nanoparticles were specifically imaged by exciting either the single peak near CE (i.e., 600 Hz below CE) with a narrow bandwidth (900 Hz) or the triplet (i.e., 2000 Hz from CE), which allowed a wider excitation bandwidth (e.g., 2018 Hz). Signal-to-noise analysis was performed on the images at the MRI console using manual regions of interest placement, and spectroscopic analysis utilized standard software provided with the scanner. Statistical comparisons were made using SAS/STAT software (SAS Co., Cary, N.C., USA).

### T1-Weighted Imaging

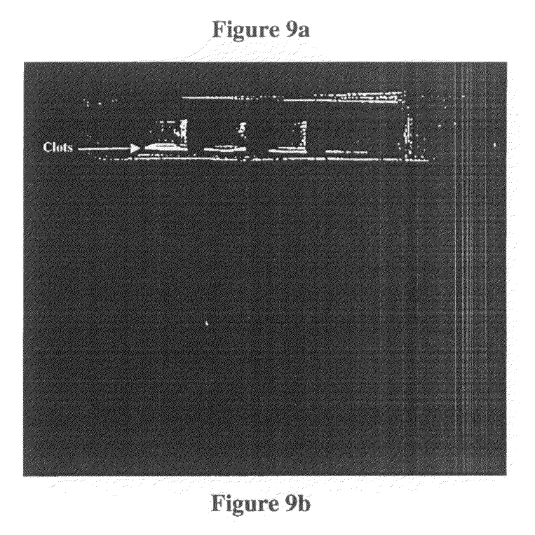

High-resolution, T1-weighted images show fibrin clots on nitrocellulose at the bottom of liquid-filled wells (see FIG. 9A). Imaging slices acquired perpendicular to the clots present a thin layer of signal enhancement from the Gd-bearing CE nanoparticles. A maximum intensity projection orthogonal to the acquisition plane (i.e., parallel to the clots) depicts the enhancement from the clots en face (FIG. 9B). As expected, signal intensity in both presentations decreased with the concentration of CE nanoparticles and concomitant increase in PFOB nanoparticles. This suggests that the nanoparticles were bound in proportions analogous to the concentrations in the original emulsion mixtures. Mean signal-to-noise ratios of the clots from each of the CE:PFOB nanoparticle mixtures, 1:0, 2:1, 1:2, and 0:1, decreased linearly: 73±5, 69±5, 63±5, and 54±4, respectively (p<0.01). Using a Look-Locker multiple echo technique, average estimates of the T1 relaxation rate (in milliseconds) of the clots were 1030±44, 1107±153, 1249±95, 1446±37, respectively, and 1524±100 for surrounding phosphate buffer solution. The three clot groups with gadolinium present were significantly different (p<0.01) from the one with no gadolinium, which was statistically the same as the surrounding solution (p>0.05).

### Spectroscopy

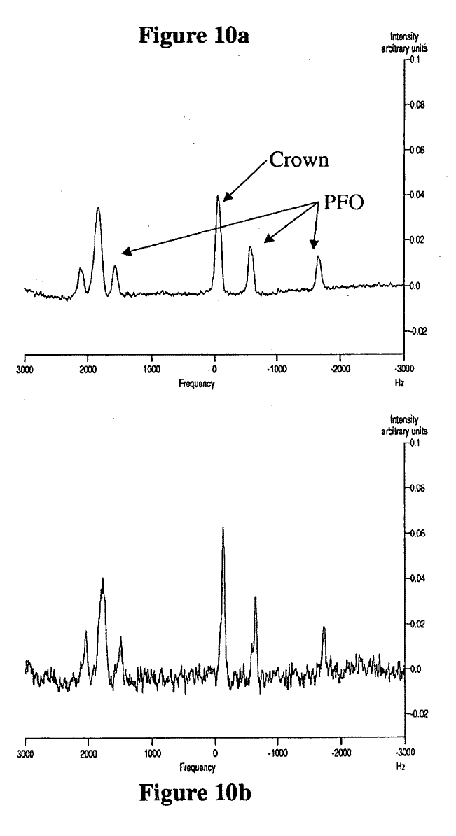

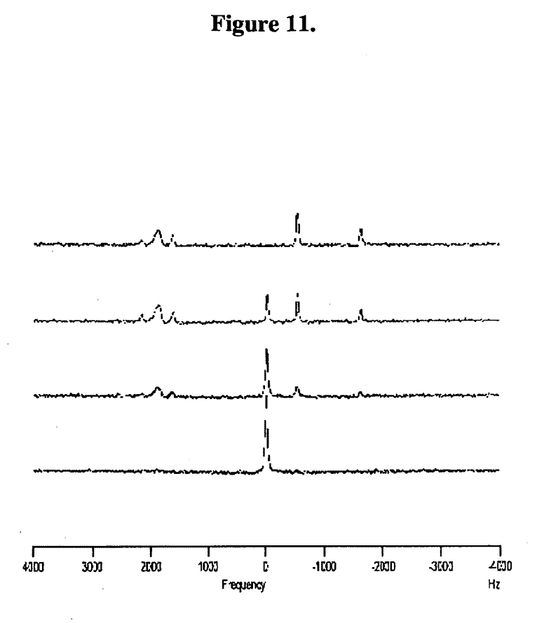

The spectrum acquired with all 12 clots positioned within the volume coil using non-volume selective FID sampling is shown in FIG. 10. Panel ‘A’ illustrates a high signal-to-noise acquisition with 472 averages; panel ‘B’ depicts a more “clinically-relevant” scan with only 16 signal averages. While somewhat noisy, this one-minute scan clearly identifies all expected peaks associated with PFOB and CE confirming the presence of both types of nanoparticles. Note that the offset frequency (i.e., 0 ppm) is set relative to the CE peak and the acquisition bandwidth of 8 kHz is ample to include all peaks (one from CE, two singlets and a triplet from PFOB). Employing ISIS spatial localization techniques, spectroscopy acquisition volumes were chosen to encompass all three clots from each of the four mixtures of nanoparticles. Shown in FIG. 11, the four volume-selective spectra reflect the varying concentration of the two perfluorocarbons as expected. Performing analysis on the CE peak (0 Hz offset) and two of the PFOB peaks (535 Hz and 1620 Hz offsets) demonstrates the area under the respective peaks changing as a function of corresponding PFC concentration (see Table II).

TABLE II
Spectral analysis of perfluorocarbon (PFC) nanoparticles bound to
clots in various relative concentrations of crown ether (CE) and
perfluorooctylbromide (PFOB).
AREA UNDER THE PEAK

PFC Concentrations in Mixture
(CE:PFOB)

| PFC Type: | 1:0 | 2:1 | 1:2 | 0:1 |
|---|---|---|---|---|
| CE | 1.63 | 1.06 | 0.470 | −0.104 |
| PFOB1 | −0.084 | 0.179 | 0.476 | 0.611 |
| PFOB2 | −0.063 | 0.030 | 0.232 | 0.343 |
| Σ PFOB | −0.147 | 0.209 | 0.708 | 0.954 |

### Spectroscopic Imaging

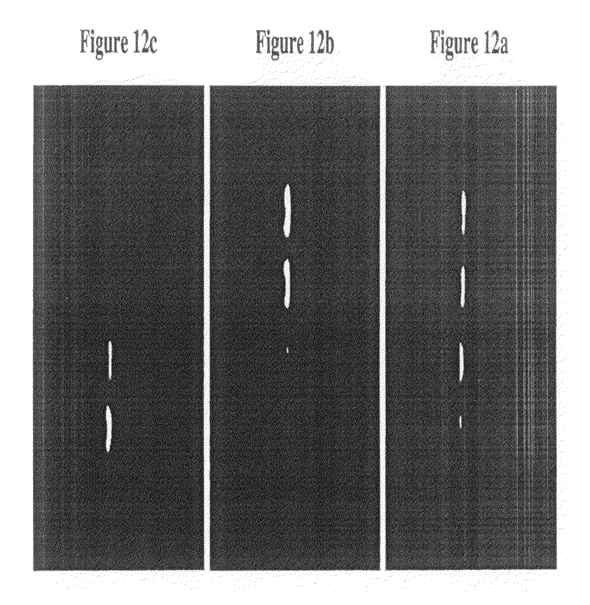

Rather than imaging based on the resonance frequency of water, images were acquired (using the same secondary transmit/receive channel dedicated to both ¹⁹F spectroscopy and imaging) of the fluorine nuclei present in the core of the fibrin-bound nanoparticles. Selecting the center frequency corresponding to that of CE and using a generous B₁ excitation bandwidth (i.e., 2 kHz) allowed the visualization of the perfluorocarbon cores bound to the clots regardless of PFC type. Narrowing the excitation bandwidth around specific center frequencies allowed selective excitation and independent visualization of the bound nanoparticles dependent on the type of PFC present in their core. FIG. 12 shows ¹⁹F images obtained, in the same orientation as in FIG. 9A, as projections through all three clots with the four varying mixtures of PFC concentrations. The first panel (A) reveals the nanoparticles on all four clot types. The next two panels, however, illustrate the results of selective excitation based on CE signature (panel B) or PFOB signature (panel C). To isolate PFOB, imaging was performed successfully by choosing either the triplet (offset 2 kHz from CE) or the singlets (e.g., offset 600 Hz from CE). Imaging on the PFOB triplet allowed a wider B₁ bandwidth than did the larger PFOB singlet, due to the proximity of the CE peak.

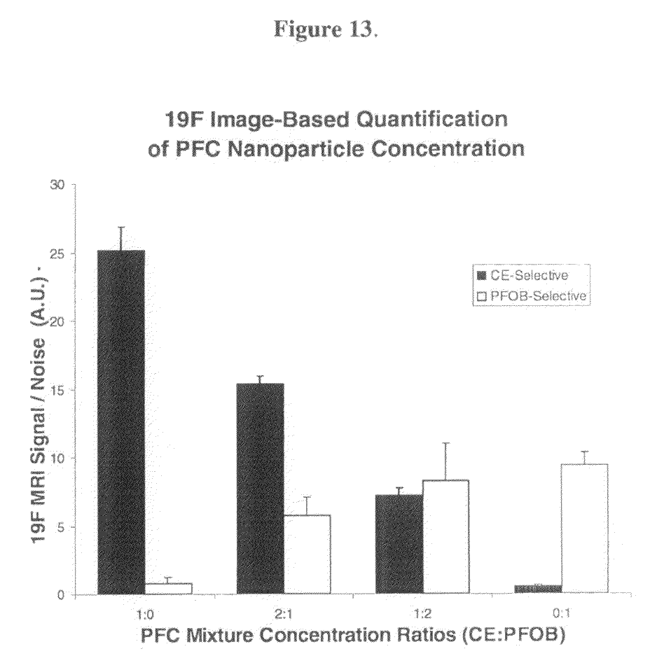

Analogous to integrating the area under the peaks of the volume selective spectra, evaluating signal to noise ratios from the various selective imaging provides an estimate of nanoparticle PFC concentration. FIG. 13 shows the deconvolution of the total signal into component parts based on the type of nanoparticle present. Fitting a linear regression line to the SNR as a function of percentage PFC concentration gives, for CE and PFOB, respectively, y=24.6x−0.248 (R²=0.993) and y=8.53x+1.78 (R²=0.917).

This example thus shows that ¹⁹F nuclei of site-targeted PFC nanoparticles can be measured not only with spectroscopy, but also imaged using a 1.5 T clinical system with short scan times based on steady-state methods. With both techniques, the detection and “deconvolution” of two different PFCs were achieved. Moreover, relative concentrations of the type of nanoparticle bound to the fibrin clots were demonstrated by spectroscopic analysis as well as simple region of interest analysis of the PFC-selective 19F images, which showed a linear relationship between signal intensity and perfluorocarbon concentration. In these three-minute images, the overall signal-to-noise of PFOB was less than that of CE, presumably due to the concentration of the signal into a single peak for CE whereas for PFOB it was distributed to multiple peaks, yet only single peaks (or triplets) were used for imaging. While the techniques are not yet fully optimized, this proof of principle supports the potential of performing quantitative ¹⁹F spectroscopy and imaging in the clinic.

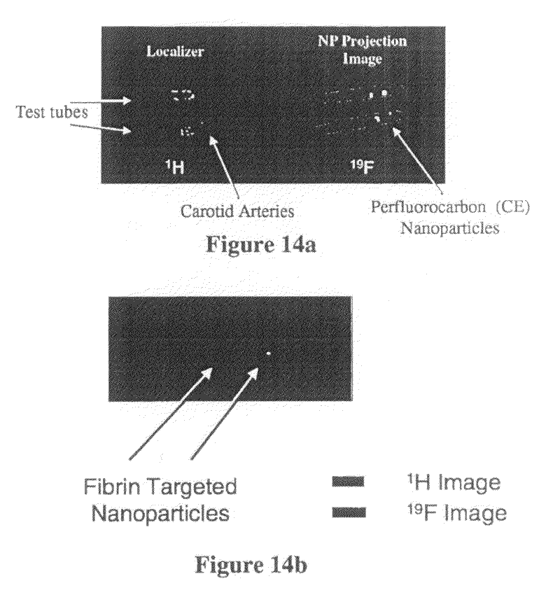

This emerging opportunity is illustrated by the imaging of two human carotid endarterectomy samples. The samples were inoculated with fibrin-targeted CE nanoparticles and rinsed (methods similar to the clots). Shown in FIG. 14A are the T1-weighted images showing the carotid samples in test tubes. In the adjacent panel, the ¹⁹F image reveals the nanoparticles bound to the exposed fibrin in the diseased carotid artery with no background signal. Combining the proton image and the fluorine image allows sensitive detection of the nanoparticles in combination with high-resolution proton imaging for anatomy. From this in vitro example, one might envision a clinical scenario where lumenally exposed microthrombi on ruptured atherosclerotic plaques might be detected, imaged, and quantified.

Furthermore, combining drug delivery and MR molecular imaging may permit phenotypic characterization of patients leading to individually matched therapy (Lanza G M, et al. Circulation. Nov. 26 2002; 106(22):2842-2847; Lanza G M, et al. Curr Pharm Biotechnol. December 2004; 5(6):495-507). As already demonstrated in early atherosclerosis (Winter P M, et al. Molecular Imaging. July 2004; 3(3):188), high resolution T1-weighted proton imaging allows visualization of targeted drug delivery with concomitant estimation of local drug concentration, i.e., rational drug dosing. Of course, as with all T1-weighted proton imaging, signal intensity can be influenced by many more factors than just site targeted accumulation of Gd. On the other hand, ¹⁹F imaging and spectroscopy provide not only quantitative signal, which is directly related to concentration and has no endogenous background, but also multi-spectral discrimination. The quantitative signal is reflective of the mass of PFC (and therefore drug or other “payloads”) deposited within a voxel. The multi-spectral capability will allow the use of multiple nanoparticles containing spectrally distinct PFCs to be targeted simultaneously, potentially to multiple sites, and measured independently as demonstrated in FIG. 11.

### Example 7

### Labelling of Monocytes with PFC Nanoparticles

A method for labeling monocytes with perfluorocarbon nanoparticles to track and quantify these cells in vivo is also provided. The collection and labeling protocol is described below and is done without the use of transfection agents in a relatively short period of time (3 hours). Monocytes and macrophages are involved in a wide variety of physiologic and pathologic processes including inflammation, atherosclerosis, and angiogenesis making the ability to accurately track and quantify them potentially clinically significant. In this particular example, murine monocytes are labelled. However, labeling of monocytes from other mammalian sources such as humans, dogs, cats, rats, rabbits, pigs, cows, horses, or monkeys is also contemplated.

To obtain the monocyte cells for labeling, the mononuclear cell fraction is separated from either peripheral blood or bone marrow using Ficoll density gradient centrifugation. The cells are then suspended in basic media (DMEM+10% FBS) and incubated with a 1:50 dilution of nanoparticle emulsion a rocker in an incubator for 3 hours. At the end of this time the cell/nanoparticle solution is centrifuged over Optiprep to remove free particles that have not been taken into cells. The remaining cells are collected as the buffy-coat, washed with PBS (Phosphate Buffered Saline) and used without further culture.

More specifically, monocyte cells for labeling have been obtained by first diluting peripheral blood 1:1 with PBS. The diluted blood is then layered over 3 mL Ficoll in a 15 mL centrifuge tube. This tube is then centrifuged at 400 g for 30 minutes at 18 degrees Centrigrade. The buffy coat from the tube is then collected and diluted to 10 mL total volume with PBS. The cells in this buffy coat are collected by centrifugation at 300 g for 15 minutes to yield a pellet of cells. This pellet is then suspended in 4 mL basic media (DMEM+10% FBS).

To label the isolated monocytes, a 1:50 volume of nanoparticle emulsion is added to cell suspension in a 2 mL centrifuge tube. This mixture is then incubated on a rocker at 34 deg and 5% CO2 for 3 hours. Following this incubation, the cell/nanoparticle solution is diluted to 5 mL with PBS and layered layer over 2.5 mL Optiprep column mixed with 50 uL 20×PBS. This column is then centrifuged at 1000 RPM (100 g) for 10 minutes. Alternatively, cell/NP solution is then centrifuged over Optiprep (specific gravity 1.32) for 20 min at 300 g to remove free NP's. Following centrifugation, the buffy coat is collected and diluted to 10 mL with PBS. Cells are then collected by centrifuging at 300 g for 15 minutes (to pellet cells). The pelleted cells are then suspended to an appropriate volume.

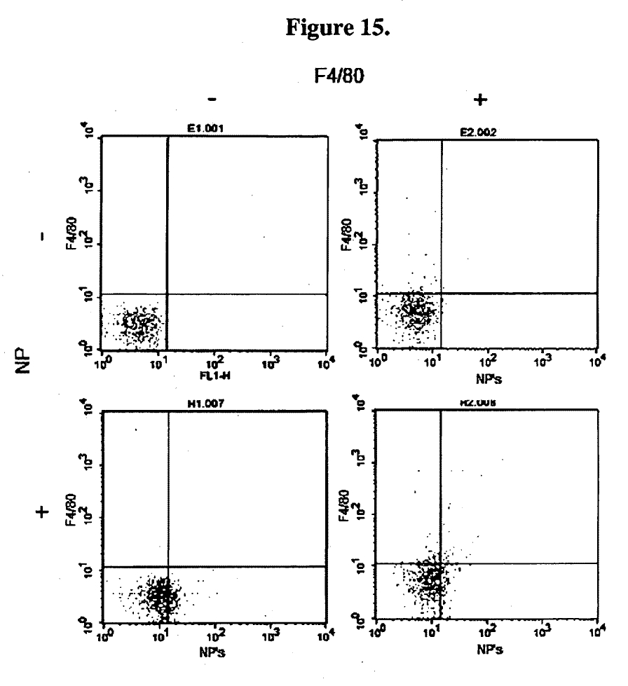

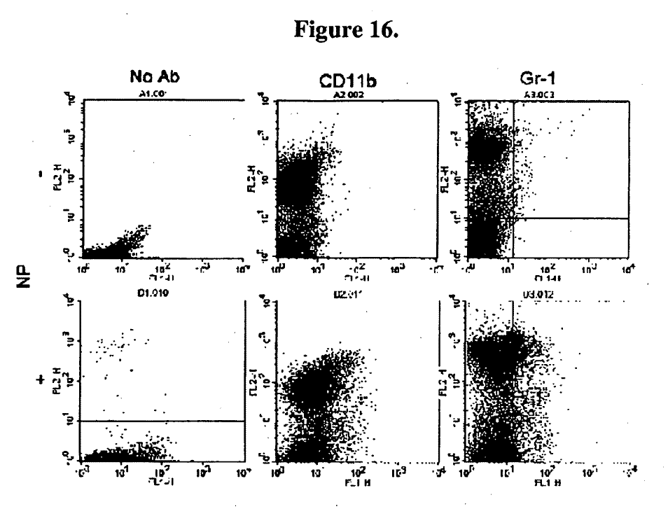

After the three hour labeling period we have observed, using flow cytometry, that cells displaying monocytic surface markers can be effectively labeled using perfluorocarbon nanoparticles. Using dye exclusion, the viability of the cells after the completion of labeling has been shown to be approximately 95%. In cells derived from the peripheral blood of mice this technique has been shown to definitively label over 40% of F4/80 positive cells (FIG. 15). F4/80 is a surface marker specific for monocytes. Additionally, in cells derived from mouse bone marrow 18% of CD11b+ cells and 15% of Gr-1+ cells were labeled (FIG. 16). Although both of these surface markers include monocytes, they are not specific and include a variety of other cells types. F4/80 cannot be used as a cell marker in this case because it is not expressed by monocytic cells derived from bone marrow. Using fluorine MR spectroscopy as few as 50,000 cells can be detected in vitro. Approximately 5-10 times this number of cells would have to be present per voxel to be imaged.

### Example 8

### Labeling of Cells with Non-Targeted PFC Nanoparticles with Ultrasound

The use of ultrasound to reduce the prolonged labeling time of up to 12 hours that is associated with labeling cells with perfluorocarbon nanoparticles (PFC NP: of ˜200 nm) is demonstrated in this Example.

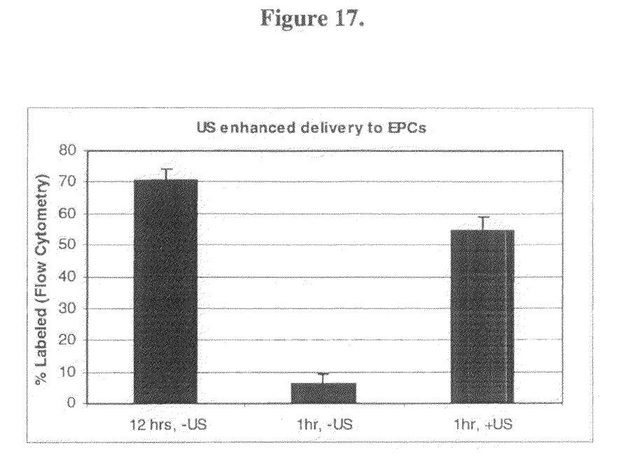

Human umbilical cord blood was obtained from the Cardinal Glennon Children's Hospital (St. Louis, Mo.) and used in accordance with ethical guidelines and accepted human studies protocols at Washington University School of Medicine. Mononuclear cells (MNCs) were isolated by density gradient centrifugation with Ficoll-Paque Plus (Amersham Biosciences, Piscataway, N.J.). MNCs were plated at concentrations of 5×10⁵ cells/cm² on fibronectin-coated T-75 plates (BD Biosciences, Franklin Lakes, N.J.) and grown in modified endothelial growth media (Clonetics EGM-2+20% FBS; Cambrex, East Rutherford, N.J.) designed to promote differentiation along the endothelial lineage. At day 2, non-adherent cells were removed and transferred to fibronectin-coated OptiCell™ cassettes (Biocrystal, Westerville, Ohio), where media was replaced every 4 days. Cassettes were coated by incubating with 2 μg/cm2 human fibronectin (Chemicon, Temechula, Calif.) for 1 hr and blocking with 2% (w/v) bovine serum albumin (Sigma, St. Louis, Mo.) for 30 min. After 7-14 days in culture, the differentiated stem/progenitor (CD34+CD133+CD31+) cells were exposed to a 30 pM concentration of perfluorocarbon nanoparticles in cell culture medium and placed in a heated (37° C.) waterbath elevated from the bottom surface. Ultrasound application was conducted with a clinical medical imager (Acuson Sequoia™; Siemens, Malvern, Pa.) with a broadband (2-3.5 MHz, 3V2a) phased-array transducer, which was applied at a slight angle approximately 2 cm from the cassette membranes. The US transducer or probe provides an ultrasound delivery field of about 2 cm² and was moved continuously across the entire surface of the cassette by a motor to expose the entire surface of the cassette (65 mm×74.8 mm) in order to transmit calibrated levels of ultrasound energy. Parameters for ultrasound delivery were a Mechanical Index (i.e. MI: the machine power output setting) of 1.9, a frequency of 2 MHz, and a focal zone setting of 20 mm. The US transducer was continuously moved across the entire surface of the cassette for a total scan time of 60 minutes, resulting in cell ultrasound exposure times of between about 1 and about 15 minutes. Flow cytometry revealed that the use of ultrasound (US) energy significantly increased the numbers of cells that are labeled (FIG. 17). In a comparison of cells labeled in the presence and absence of US for a 1 hour period, 55±4% of the treated cells were labelled when ultrasound energy was used while only 6±3% respectively of the cells that were not treated with ultrasound were labelled (p<0.001). The observed 55% labeling efficiency obtained in a one hour period with ultrasound approaches the 71.4% labeling efficiency that is achieved in the 12 hour incubation method that does not employ ultrasound. No untoward effects of the ultrasound labeling procedure on cell viability were observed by trypan blue dye exclusion when comparing untreated and US-exposed cells (both methods showed that ˜90% of the cells were viable following labeling). This unique approach should facilitate investigation of regenerative therapeutics by providing a safe adjunctive method to label cells for real-time tracking. Furthermore, the labeling procedures can be carried out with conventional ultrasound imaging devices available to most researchers.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

Each patent and non-patent documents cited herein is expressly incorporated herein by reference in its entirety.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. A method for obtaining an endothelial precursor cell suitable for magnetic resonance imaging comprising the steps of:
a. providing an endothelial precursor cell;
b. incubating said endothelial precursor cell in a cell culture media containing a plurality of perfluorocarbon nanoparticles for a period of time and at a perfluorocarbon nanoparticle concentration sufficient to result in internalization of a detectable level of perfluorocarbon nanoparticles; and
c. separating said endothelial precursor cell from step (b) from said culture media containing perfluorocarbon nanoparticles;
thereby obtaining an endothelial precursor cell suitable for magnetic resonance imaging.

2. The method of claim 1, wherein said plurality of perfluorocarbon nanoparticles comprise a perfluorooctylbromide core component or a perfluoro-15-crown-5-ether core component.

3. The method of claim 2, wherein said plurality of perfluorocarbon nanoparticles comprise a perfluorooctylbromide core component and wherein said detectable level of internalized perfluorocarbon nanoparticles is an intracellular perfluorocarbon nanoparticle concentration of at least 2.8 pmol per cell.

4. The method of claim 2, wherein said plurality of perfluorocarbon nanoparticles comprise a perfluoro-15-crown-5-ether core component and wherein said detectable level of internalized perfluorocarbon nanoparticles is an intracellular perfluorocarbon nanoparticle concentration of at least 0.5 pmol per cell.

5-8. (canceled)

9. The method of claim 1, wherein said concentration of perfluorocarbon nanoparticles is at least about 30 pM.

10-13. (canceled)

14. The method of claim 1, wherein said endothelial precursor cell suitable for magnetic resonance imaging can internalize acetylated-Low Density Lipoprotein (LDL) and wherein fucose is present at the surface of said endothelial precursor cell suitable for magnetic resonance imaging.

15. (canceled)

16. The method of claim 1 wherein each perfluorocarbon nanoparticle in said a plurality of perfluorocarbon nanoparticles used in step (b) have an average diameter in a range of about 200 nm to about 300 nm.

17-29. (canceled)

30. A method of obtaining two distinct magnetic resonance imaging data sets derived from two distinct cells introduced into a system, comprising the steps of:
a) obtaining a first cell containing a first intracellular perfluorocarbon nanoparticle, wherein said first intracellular perfluorocarbon nanoparticle comprises a perfluoro-15-crown-5-ether core component and wherein said first intracellular perfluorocarbon nanoparticle is at a detectable level in said first cell;
b) obtaining a second cell containing a second intracellular perfluorocarbon nanoparticle, wherein said second intracellular perfluorocarbon nanoparticle comprises a perfluorooctylbromide core component and wherein said second intracellular perfluorocarbon nanoparticle is at a detectable level in said second cell;
c) introducing said first cell from step (a) and said second cell from step (b) into a system;
d) exposing said system from step (c) to a first magnetic field and obtaining magnetic resonance imaging data for said first cell with a magnetic resonance imaging method that specifically detects a first ¹⁹F MRI signal from said perfluoro-15-crown-5-ether core component to obtain a first imaging data set from said first cell,
e) exposing said system from step (c) to a second magnetic field and obtaining magnetic resonance imaging data for said second cell with a magnetic resonance imaging method that specifically detects a second ¹⁹F MRI signal from said perfluorooctylbromide core component to obtain a second imaging data set from said second cell, thereby obtaining two distinct magnetic resonance imaging data sets derived from two distinct cells introduced into a system.

31. The method of claim 30, wherein said first cell in step (a) containing said first intracellular perfluorocarbon nanoparticle is obtained by introducing said first perfluorocarbon nanoparticles comprising a perfluoro-15-crown-5-ether core component into said cells by a method selected from the group consisting of electroporation, transfection, ultrasound, and sonication and wherein said second cell in step (b) containing said second intracellular perfluorocarbon nanoparticle is obtained by introducing said second perfluorocarbon nanoparticle comprising said perfluorooctylbromide core component into said cells by a method selected from the group consisting of electroporation, transfection, ultrasound, and sonication.

32-40. (canceled)

41. The method of claim 30, wherein said system is an in vitro system or a living organism.

42-46. (canceled)

47. The method of claim 30, wherein said first magnetic field in step (d) and said second magnetic field in step (e) have a field strength 11.7 T or of 1.5 T.

48. (canceled)

49. The method of claim 30, wherein said magnetic resonance imaging method in step (d) that specifically detects a first ¹⁹F MRI signal from said perfluoro-15-crown-5-ether core component comprises use of an excitation signal centered at a frequency that is substantially the same as the resonance frequency of said perfluoro-15-crown-5-ether core component, wherein said excitation signal bandwidth does not overlap any of several resonance frequencies of said perfluorooctylbromide core component of step (b).

50-51. (canceled)

52. The method of claim 30, wherein said magnetic resonance imaging method in step (e) that specifically detects a second ¹⁹F MRI signal from said perfluorooctylbromide core component comprises use of an excitation signal centered at a frequency that is substantially the same as the resonance frequency of at least one selected spectral peak generated by the perfluorooctylbromide core component, wherein said excitation signal bandwidth does not overlap the resonance frequency of said perfluoro-15-crown-5-ether core component of step (a).

53-55. (canceled)

56. A method of obtaining two distinct magnetic resonance spectroscopy data sets derived from two distinct cells introduced into a system, comprising the steps of:
a) obtaining a first cell containing a first intracellular perfluorocarbon nanoparticle, wherein said first intracellular perfluorocarbon nanoparticle comprises a perfluoro-15-crown-5-ether core component and wherein said first intracellular perfluorocarbon nanoparticle is at a detectable level of said first cell;
b) obtaining a second cell containing a second intracellular perfluorocarbon nanoparticle, wherein said second intracellular perfluorocarbon nanoparticle comprises a perfluorooctylbromide core component and wherein said second intracellular perfluorocarbon nanoparticle is at a detectable level in said second cell;
c) introducing said first cell from step (a) and said second cell from step (b) into a system;
d) exposing said system from step (c) to a first magnetic field and obtaining magnetic resonance spectroscopy data for said first cell with a magnetic resonance spectroscopy method that specifically detects a first ¹⁹F MRI signal from said perfluoro-15-crown-5-ether core component to obtain a first spectroscopy data set from said first cell,
e) exposing said system from step (c) to a second magnetic field and obtaining magnetic resonance spectroscopy data for said second cell with a magnetic resonance spectroscopy method that specifically detects a second ¹⁹F MRI signal from said perfluorooctylbromide core component to obtain a second spectroscopy data set from said second cell, thereby obtaining two distinct magnetic resonance spectroscopy data sets derived from two distinct cells introduced into a system.

57. The method of claim 56, wherein said first cell in step (a) containing said first intracellular perfluorocarbon nanoparticle is obtained by introducing said first perfluorocarbon nanoparticles comprising a perfluoro-15-crown-5-ether core component into said cells by a method selected from the group consisting of electroporation, transfection, ultrasound, and sonication and wherein said second cell in step (b) containing said second intracellular perfluorocarbon nanoparticle is obtained by introducing said second perfluorocarbon nanoparticle comprising said perfluorooctylbromide core component into said cells by a method selected from the group consisting of electroporation, transfection, ultrasound, and sonication.

58-66. (canceled)

67. The method of claim 56, wherein said system is an in vitro system or a living organism.

68-72. (canceled)

73. The method of claim 56, wherein said first magnetic field in step (d) and said second magnetic field in step (e) have a field strength of 11.7 T or of 1.5 T.

74. (canceled)

75. The method of claim 56, wherein obtainment of magnetic resonance spectroscopy data in steps (d) and (e) comprises acquisition of volume selective spectra by image-selective in vivo spectroscopy.

76-79. (canceled)

80. A method for obtaining a cell suitable for magnetic resonance imaging comprising the steps of:
a. providing at least one cell;
b. treating said cell or cells in a cell culture media containing a plurality of non-targeted perfluorocarbon nanoparticles with ultrasound energy for a period of time and at a perfluorocarbon nanoparticle concentration sufficient to result in internalization of a detectable level of perfluorocarbon nanoparticles; and
c. separating said cell from step (b) from said culture media containing perfluorocarbon nanoparticles; thereby obtaining a cell suitable for magnetic resonance imaging.

81-83. (canceled)

84. The method of claim 80, wherein said concentration of perfluorocarbon nanoparticles in step (b) is at least about 30 pM.

85. (canceled)

86. The method of claim 80, wherein said cells in step (a) are distributed across a surface and wherein ultrasound energy is delivered to a plurality of fields within said surface.

87. The method of claim 80, wherein said cells in step (a) are distributed in distinct wells of a microtiter plate and wherein ultrasound energy is delivered to individual wells of said microtiter plate.
